(19) United States
(12) Patent Application Publication
(10) Pub. No.: US 2023/0380719 A1
(43) Pub. Date: Nov. 30, 2023

(54) METHOD AND APPARATUS FOR SIMULTANEOUS COLLECTION, PROCESSING AND DISPLAY OF AUDIO AND FLOW EVENTS DURING BREATHING

(71) Applicant: AireHealth Inc., Winter Springs, FL (US)

(72) Inventors: Charalampos-Christos Stamatopoulos, Athens (GR); Francis Patrick O'Neill, Kissimmee, FL (US)

(21) Appl. No.: 17/829,081

(22) Filed: May 31, 2022

(51) Int. Cl. A61B 5/087; A61B 5/097; A61B 5/091; A61B 5/00

(52) U.S. Cl. CPC A61B 5/087; A61B 5/097; A61B 5/091; A61B 5/7267; A61B 5/7289; A61B 5/743

(57) ABSTRACT

A computer-implemented method for determining lung pathology from audio respiratory and breath flow signals comprises receiving a plurality of breath flow signals and a plurality of audio signals comprising a training set for a convolutional neural network (CNN), wherein the plurality of breath flow signals and the plurality of audio signals are extracted from sessions with patients with known pathologies of known degrees of severity. The method also comprises analyzing the plurality of audio signals and the plurality of breath flow signals, wherein the analyzing comprises extracting a plurality of descriptors associated with the audio and breath flow signals. Further, the method comprises creating a plurality of graphs using information from the descriptors, wherein at least one of the graphs comprises a plot combining descriptors from both the audio and the breath flow signals. The method also comprises training the CNN using the plurality of graphs.

### FIELD OF THE INVENTION

Embodiments according to the present invention relate to dynamically analyzing breathing sounds and breath flow volume using an electronic device.

### BACKGROUND OF THE INVENTION

Conventional respiratory analysis systems are capable of recording respiratory audio associated with a patient, analyzing the audio and providing feedback regarding possible pathologies from which the patient may be suffering. The principal drawback of conventional respiratory analysis systems is that only part of the available information collected from the patient is collected, processed, and displayed. Accordingly, conventional respiratory analysis systems are unable to process events and information that would lead to a deeper understanding of the pathology and the manner in which a particular disease or pathology progresses over time.

Another drawback of conventional respiratory analysis systems is that the framework and processes for determining pathologies are not optimized for determining the manner in which a condition or a pathology is trending over time. Accordingly, a deeper understanding of how a pathology is responding to treatments or changing over time is unavailable.

### BRIEF SUMMARY OF THE INVENTION

Accordingly, there is a need for improved methods and apparatus to process events and information associated with audio respiratory signals in a way that provides deeper insight into a patient's pathology and the manner in which the pathology is progressing over time.

Embodiments of the present invention use respiratory audio data in conjunction with breath volume and flow data to gain a deeper understanding of a patient's pathology and the manner in which a particular disease or pathology progresses over time. In one embodiment, both audio signals and breath flow are captured by a dual or multi-sense spirometer.

Capturing breath flow in conjunction with audio signals provides distinct advantages. Analysis of breath flow and audio signals collected simultaneously may be used to suppress ambient noise. Audio in the absence of any detected breath flow is likely ambient noise. The ambient noise captured from the audio signal in the absence of any breath flow can be filtered out of the audio signal to improve signal strength and integrity.

More importantly, however, flow and audio signals collected simultaneously allows the spirometer to extract custom features that are descriptive of breathing quality but also of respiratory pathology severity. In other words, flow/volume signals in conjunction with audio signals advantageously provide unique insight into patient pathology and severity that could not be extracted from the respiratory audio signal alone. Furthermore, the combination of the flow/volume signals and audio signals allows descriptor and description combinations to be extracted that were not possible using only the sound-based extraction methods.

In one embodiment, a computer-implemented method for determining lung pathology from audio respiratory and breath flow signals is disclosed. The method comprises receiving a plurality of breath flow signals and a plurality of audio signals comprising a training set for a convolutional neural network, wherein the plurality of breath flow signals and the plurality of audio signals are extracted from sessions with patients with known pathologies of known degrees of severity. The method further comprises collecting the flow and sound signals in a synchronized fashion or automatically synchronizing each of the plurality of breath flow signals with a corresponding one of the plurality of audio signals. Also, the method comprises analyzing the plurality of audio signals and the plurality of breath flow signals, wherein the analyzing comprises extracting a plurality of descriptors associated with the plurality of audio signals and the plurality of breath flow signals. Additionally, the method comprises creating a plurality of graphs using information from the plurality of descriptors, wherein at least one of the graphs comprises a plot combining descriptors from both the plurality of audio signals and the plurality of breath flow signals and training the convolutional neural network using the plurality of graphs. The method also comprises creating at least one image using a breath flow signal and an audio signal from a new patient and inputting the at least one image associated with the new patient into the convolutional neural network. Finally, the method comprises determining a pathology and associated severity for the new patient using the convolutional neural network.

In one embodiment, what is disclosed is a non-transitory computer-readable storage medium having stored thereon, computer executable instructions that, if executed by a computer system cause the computer system to perform a method determining lung pathology from audio respiratory and breath flow signals The method comprises receiving a plurality of breath flow signals and a plurality of audio signals comprising a training set for a convolutional neural network, wherein the plurality of breath flow signals and the plurality of audio signals are extracted from sessions with patients with known pathologies of known degrees of severity. The method further comprises collecting the flow and sound signals in a synchronized fashion or synchronizing each of the plurality of breath flow signals with a corresponding one of the plurality of audio signals. Also, the method comprises analyzing the plurality of audio signals and the plurality of breath flow signals, wherein the analyzing comprises extracting a plurality of descriptors associated with the plurality of audio signals and the plurality of breath flow signals. Additionally, the method comprises creating a plurality of graphs using information from the plurality of descriptors, wherein at least one of the graphs comprises a plot combining descriptors from both the plurality of audio signals and the plurality of breath flow signals and training the convolutional neural network using the plurality of graphs. The method also comprises creating at least one image using a breath flow signal and an audio signal from a new patient and inputting the at least one image associated with the new patient into the convolutional neural network. Finally, the method comprises determining a pathology and associated severity for the new patient using the convolutional neural network.

In one embodiment, a device for determining lung pathology from breath flow and audio respiratory signals is disclosed. In one embodiment, the device may be a spirometer. The device comprises a memory for storing a plurality of audio signals and a plurality of breath flow signals, instructions associated with a convolutional neural network and a process for determining lung pathology from the plurality of audio signals and the plurality of breath flow signals. The spirometer further comprises a processor coupled to the memory, the processor being configured to operate in accordance with the instructions to: a) receive a plurality of breath flow signals and a plurality of audio signals comprising a training set for a convolutional neural network, wherein the plurality of breath flow signals and the plurality of audio signals are extracted from sessions with patients with known pathologies of known degrees of severity; b) collect the flow and sound signals in a synchronized fashion or synchronize each of the plurality of breath flow signals with a corresponding one of the plurality of audio signals; c) analyze the plurality of audio signals and the plurality of breath flow signals, wherein the analyzing comprises extracting a plurality of descriptors associated with the plurality of audio signals and the plurality of breath flow signals; d) create a plurality of graphs using information from the plurality of descriptors, wherein at least one of the graphs comprises a plot combining descriptors from both the plurality of audio signals and the plurality of breath flow signals; e) train the convolutional neural network using the plurality of graphs; f) create at least one image using a breath flow signal and an audio signal from a new patient; g) input the at least one image associated with the new patient into the convolutional neural network; and h) determine a pathology and associated severity for the new patient using the convolutional neural network.

The following detailed description together with the accompanying drawings will provide a better understanding of the nature and advantages of the present invention.

### BRIEF DESCRIPTION OF THE DRAWINGS

Embodiments of the present invention are illustrated by way of example, and not by way of limitation, in the figures of the accompanying drawings and in which like reference numerals refer to similar elements.

FIG. 1 is an exemplary computer system in accordance with embodiments of the present invention.

FIG. 2 illustrates a user breathing into an exemplary apparatus comprising a microphone for capturing breathing sounds and a volume sensor for capturing flow signals in accordance with an embodiment of the present invention.

FIG. 3 illustrates an exemplary apparatus comprising a microphone for capturing breathing sounds and a volume sensor for capturing flow signals in accordance with the methods and apparatus of embodiments of the present invention.

FIG. 4A illustrates an exemplary flow diagram indicating the manner in which a dynamic respiratory classifier and tracker (DRCT) framework can be used in evaluating lung pathology in accordance with an embodiment of the present invention.

FIG. 4B illustrates an exemplary flow diagram indicating the manner in which the DRCT framework can be used in evaluating lung pathology where inputs are received from several different types of sensors in accordance with an embodiment of the present invention.

FIG. 5 illustrates a spirometer with built-in lung sound analysis in accordance with an embodiment of the present invention.

FIG. 6A illustrates a data flow diagram of a process that can be implemented to extract spectrograms and sound-based descriptors pertaining to wheeze in accordance with an embodiment of the present invention.

FIG. 6B illustrates a data flow diagram of a process that can be implemented to extract sound based descriptors pertaining to crackling in accordance with an embodiment of the present invention.

FIG. 7 depicts a flowchart illustrating an exemplary computer-implemented process for detecting the wheeze start time in accordance with one embodiment of the present invention.

FIG. 8 depicts a flowchart illustrating an exemplary computer-implemented process for determining wheeze source in accordance with one embodiment of the present invention.

FIG. 9A is an exemplary spectrogram associated with the wheezing behavior of a hypothetical subject in accordance with an embodiment of the present invention.

FIG. 9B illustrates an exemplary magnified spectrogram associated with the wheezing behavior of a hypothetical subject in accordance with an embodiment of the present invention.

FIG. 10A illustrates an exemplary spectrogram associated with the wheezing behavior of a hypothetical subject in accordance with an embodiment of the present invention.

FIG. 10B illustrates an exemplary magnified spectrogram which is a magnified version of the spectrogram shown in FIG. 10A in accordance with an embodiment of the present invention.

FIG. 10C illustrates a wheeze-only spectrogram associated with the wheezing behavior of a hypothetical subject shown in FIG. 10A in accordance with an embodiment of the present invention.

FIG. 11 illustrates the manner in which the filtered impulse response is created by filtering a delta function to create an artificial crackle in accordance with an embodiment of the present invention.

FIG. 12 illustrates the cross correlation function determined using the frame and the normalized filtered response in accordance with an embodiment of the present invention.

FIG. 13 illustrates a block diagram providing an overview of the manner in which an artificial neural network can be trained to ascertain lung pathologies in accordance with an embodiment of the present invention.

FIG. 14 illustrates a block diagram providing an overview of the manner in which an artificial neural network can be used to evaluate a respiratory recording associated with a patient to determine lung pathologies and severity in accordance with an embodiment of the present invention.

FIG. 15 illustrates exemplary original spectrogram PDFs aggregated over pathology and severity in accordance with an embodiment of the present invention.

FIG. 16 illustrates exemplary results from the binary hypothesis testing conducted at block 1405 in accordance with an embodiment of the present invention.

FIG. 17 depicts a flowchart illustrating an exemplary computer-implemented process for determining lung pathologies and severity from a respiratory recording using an artificial neural network in accordance with one embodiment of the present invention.

FIG. 18 illustrates a block diagram providing an overview of the manner in which a convolutional neural network (CNN) can be trained to ascertain lung pathologies in accordance with an embodiment of the present invention.

FIG. 19 illustrates a block diagram providing an overview of the manner in which a convolutional neural network (CNN) can be used to evaluate a respiratory recording associated with a new patient to determine lung pathologies and severity in accordance with an embodiment of the present invention.

FIG. 20 depicts a flowchart illustrating an exemplary computer-implemented process for determining lung pathologies and severity from a respiratory recording and breath flow analysis using a convolutional neural network in accordance with one embodiment of the present invention.

FIG. 21A is an exemplary illustration of a flow-over-time graph associated with a healthy patient in accordance with an embodiment of the present invention.

FIG. 21B is an exemplary illustration of a flow-over-time graph associated with an unhealthy patient in accordance with an embodiment of the present invention.

FIG. 22A is an exemplary illustration of a volume-over-time graph associated with a healthy patient in accordance with an embodiment of the present invention.

FIG. 22B is an exemplary illustration of a volume-over-time graph associated with an unhealthy patient in accordance with an embodiment of the present invention.

FIG. 23A is an exemplary illustration of a flow-volume loop associated with a healthy patient in accordance with an embodiment of the present invention.

FIG. 23B is an exemplary illustration of a flow-volume loop associated with an unhealthy patient in accordance with an embodiment of the present invention.

FIG. 24A is an exemplary illustration of a wheeze-clarity—over volume graph associated with a healthy patient in accordance with an embodiment of the present invention.

FIG. 24B is an exemplary illustration of a wheeze-clarity over volume graph associated with an unhealthy patient in accordance with an embodiment of the present invention.

FIG. 25A is an exemplary illustration of a wheeze-clarity over wheeze-frequency graph associated with a healthy patient in accordance with an embodiment of the present invention.

FIG. 25B is an exemplary illustration of wheeze-clarity over wheeze-frequency graph associated with an unhealthy patient in accordance with an embodiment of the present invention

FIG. 26A is an exemplary illustration of a wheeze-flow-intensity over flow graph associated with a healthy patient in accordance with an embodiment of the present invention.

FIG. 26B is an exemplary illustration of a wheeze-flow-intensity over flow graph associated with an unhealthy patient in accordance with an embodiment of the present invention.

FIG. 27A is an exemplary illustration of a wheeze-frequency over volume associated with a healthy patient in accordance with an embodiment of the present invention.

FIG. 27B is an exemplary illustration of a wheeze-frequency over volume graph associated with an unhealthy patient in accordance with an embodiment of the present invention

FIG. 28A is an exemplary illustration of a wheeze-intensity-flow over volume associated with a healthy patient in accordance with an embodiment of the present invention.

FIG. 28B is an exemplary illustration of a wheeze-intensity-flow over volume graph associated with an unhealthy patient in accordance with an embodiment of the present invention. These graphs are examples of images that are generated by using a combination of sound-based and flow-based descriptors.

FIG. 29A is an exemplary illustration of a wheeze-intensity over flow associated with a healthy patient in accordance with an embodiment of the present invention.

FIG. 29B is an exemplary illustration of a wheeze-intensity over flow graph associated with an unhealthy patient in accordance with an embodiment of the present invention. These graphs are examples of images that are generated by using a combination of sound-based and flow-based descriptors.

FIG. 30A is an exemplary illustration of a wheeze-intensity over volume associated with a healthy patient in accordance with an embodiment of the present invention.

FIG. 30B is an exemplary illustration of a wheeze-intensity over volume graph associated with an unhealthy patient in accordance with an embodiment of the present invention.

FIG. 31A is an exemplary illustration of a wheeze-intensity over wheeze frequency associated with a healthy patient in accordance with an embodiment of the present invention.

FIG. 31B is an exemplary illustration of a wheeze-intensity over wheeze frequency graph associated with an unhealthy patient in accordance with an embodiment of the present invention.

FIG. 32 illustrates an exemplary flow volume loop.

FIG. 33 depicts a flowchart illustrating an exemplary computer-implemented process for determining lung pathologies and severity from a respiratory recording and breath flow analysis using a convolutional neural network in accordance with one embodiment of the present invention.

In the figures, elements having the same designation have the same or similar function.

### DETAILED DESCRIPTION OF THE INVENTION

Reference will now be made in detail to the various embodiments of the present disclosure, examples of which are illustrated in the accompanying drawings. While described in conjunction with these embodiments, it will be understood that they are not intended to limit the disclosure to these embodiments. On the contrary, the disclosure is intended to cover alternatives, modifications and equivalents, which may be included within the spirit and scope of the disclosure as defined by the appended claims. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure.

Some portions of the detailed descriptions that follow are presented in terms of procedures, logic blocks, processing, and other symbolic representations of operations on data bits within a computer memory. These descriptions and representations are the means used by those skilled in the data processing arts to most effectively convey the substance of their work to others skilled in the art. In the present application, a procedure, logic block, process, or the like, is conceived to be a self-consistent sequence of steps or instructions leading to a desired result. The steps are those utilizing physical manipulations of physical quantities. Usually, although not necessarily, these quantities take the form of electrical or magnetic signals capable of being stored, transferred, combined, compared, and otherwise manipulated in a computer system. It has proven convenient at times, principally for reasons of common usage, to refer to these signals as transactions, bits, values, elements, symbols, characters, samples, pixels, or the like.

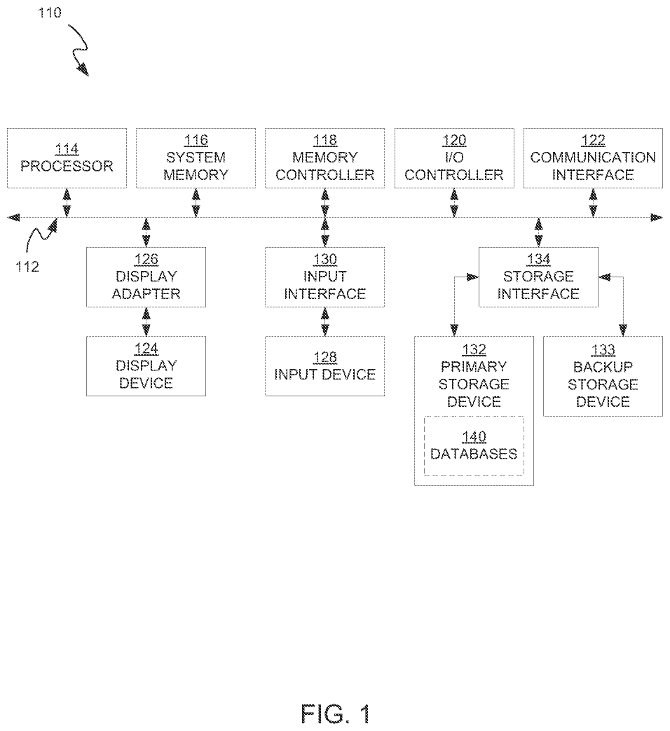

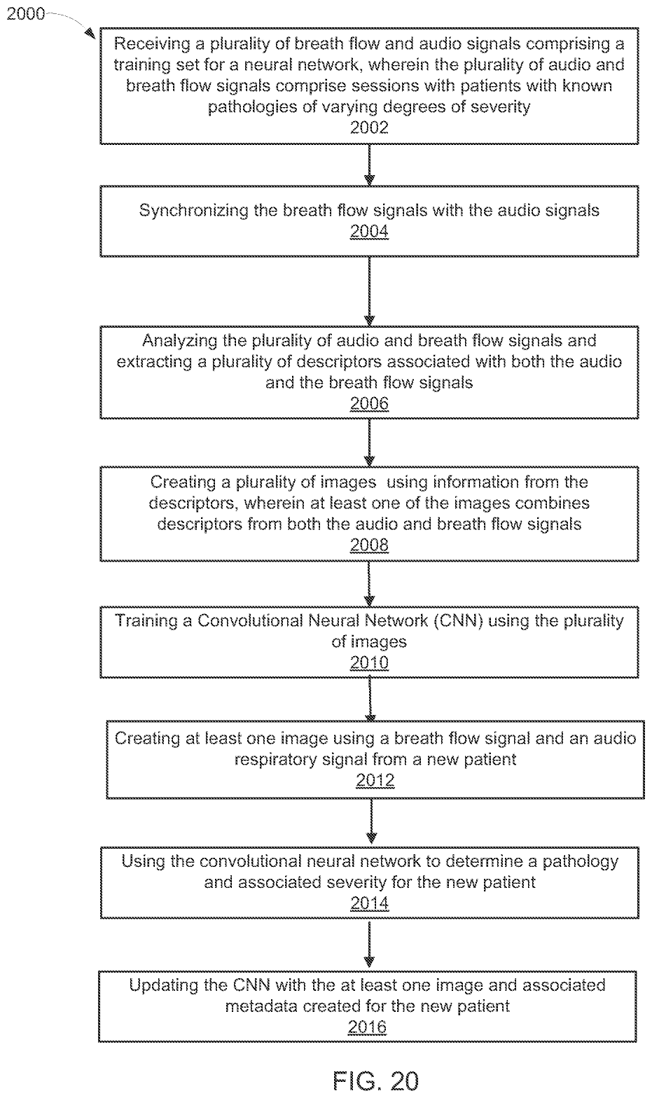

It should be borne in mind, however, that all of these and similar terms are to be associated with the appropriate physical quantities and are merely convenient labels applied to these quantities. Unless specifically stated otherwise as apparent from the following discussions, it is appreciated that throughout the present disclosure, discussions utilizing terms such as “analyzing,” “generating,” “classifying,” “filtering,” “calculating,” “performing,” “extracting,” “recognizing,” “capturing,” or the like, refer to actions and processes (e.g., flowchart 2000 of FIG. 20) of a computer system or similar electronic computing device or processor (e.g., system 110 of FIG. 1). The computer system or similar electronic computing device manipulates and transforms data represented as physical (electronic) quantities within the computer system memories, registers or other such information storage, transmission or display devices.

Embodiments described herein may be discussed in the general context of computer-executable instructions residing on some form of computer-readable storage medium, such as program modules, executed by one or more computers or other devices. By way of example, and not limitation, computer-readable storage media may comprise non-transitory computer-readable storage media and communication media; non-transitory computer-readable media include all computer-readable media except for a transitory, propagating signal. Generally, program modules include routines, programs, objects, components, data structures, etc., that perform particular tasks or implement particular abstract data types. The functionality of the program modules may be combined or distributed as desired in various embodiments.

Computer storage media includes volatile and nonvolatile, removable and non-removable media implemented in any method or technology for storage of information such as computer-readable instructions, data structures, program modules or other data. Computer storage media includes, but is not limited to, random access memory (RAM), read only memory (ROM), electrically erasable programmable ROM (EEPROM), flash memory or other memory technology, compact disk ROM (CD-ROM), digital versatile disks (DVDs) or other optical storage, magnetic cassettes, magnetic tape, magnetic disk storage or other magnetic storage devices, or any other medium that can be used to store the desired information and that can accessed to retrieve that information.

Communication media can embody computer-executable instructions, data structures, and program modules, and includes any information delivery media. By way of example, and not limitation, communication media includes wired media such as a wired network or direct-wired connection, and wireless media such as acoustic, radio frequency (RF), infrared, and other wireless media. Combinations of any of the above can also be included within the scope of computer-readable media.

FIG. 1 is a block diagram of an example of a computing system 110 used to perform respiratory acoustic analysis to track patient pathologies and capable of implementing embodiments of the present disclosure. Computing system 110 broadly represents any single or multi-processor computing device or system capable of executing computer-readable instructions. Examples of computing system 110 include, without limitation, workstations, laptops, client-side terminals, servers, distributed computing systems, handheld devices, or any other computing system or device. In its most basic configuration, computing system 110 may include at least one processor 114 and a system memory 116.

Processor 114 generally represents any type or form of processing unit capable of processing data or interpreting and executing instructions. In certain embodiments, processor 114 may receive instructions from a software application or module. These instructions may cause processor 114 to perform the functions of one or more of the example embodiments described and/or illustrated herein.

System memory 116 generally represents any type or form of volatile or non-volatile storage device or medium capable of storing data and/or other computer-readable instructions. Examples of system memory 116 include, without limitation, RAM, ROM, flash memory, or any other suitable memory device. Although not required, in certain embodiments computing system 110 may include both a volatile memory unit (such as, for example, system memory 116) and a non-volatile storage device (such as, for example, primary storage device 132).

Computing system 110 may also include one or more components or elements in addition to processor 114 and system memory 116. For example, in the embodiment of FIG. 1, computing system 110 includes a memory controller 118, an input/output (I/O) controller 120, and a communication interface 122, each of which may be interconnected via a communication infrastructure 112. Communication infrastructure 112 generally represents any type or form of infrastructure capable of facilitating communication between one or more components of a computing device. Examples of communication infrastructure 112 include, without limitation, a communication bus (such as an Industry Standard Architecture (ISA), Peripheral Component Interconnect (PCI), PCI Express (PCIe), or similar bus) and a network.

Memory controller 118 generally represents any type or form of device capable of handling memory or data or controlling communication between one or more components of computing system 110. For example, memory controller 118 may control communication between processor 114, system memory 116, and I/O controller 120 via communication infrastructure 112.

I/O controller 120 generally represents any type or form of module capable of coordinating and/or controlling the input and output functions of a computing device. For example, I/O controller 120 may control or facilitate transfer of data between one or more elements of computing system 110, such as processor 114, system memory 116, communication interface 122, display adapter 126, input interface 130, and storage interface 134.

Communication interface 122 broadly represents any type or form of communication device or adapter capable of facilitating communication between example computing system 110 and one or more additional devices. For example, communication interface 122 may facilitate communication between computing system 110 and a private or public network including additional computing systems. Examples of communication interface 122 include, without limitation, a wired network interface (such as a network interface card), a wireless network interface (such as a wireless network interface card), a modem, and any other suitable interface. In one embodiment, communication interface 122 provides a direct connection to a remote server via a direct link to a network, such as the Internet. Communication interface 122 may also indirectly provide such a connection through any other suitable connection.

Communication interface 122 may also represent a host adapter configured to facilitate communication between computing system 110 and one or more additional network or storage devices via an external bus or communications channel. Examples of host adapters include, without limitation, Small Computer System Interface (SCSI) host adapters, Universal Serial Bus (USB) host adapters, IEEE (Institute of Electrical and Electronics Engineers) 1394 host adapters, Serial Advanced Technology Attachment (SATA) and External SATA (eSATA) host adapters, Advanced Technology Attachment (ATA) and Parallel ATA (PATA) host adapters, Fibre Channel interface adapters, Ethernet adapters, or the like. Communication interface 122 may also allow computing system 110 to engage in distributed or remote computing. For example, communication interface 122 may receive instructions from a remote device or send instructions to a remote device for execution.

As illustrated in FIG. 1, computing system 110 may also include at least one display device 124 coupled to communication infrastructure 112 via a display adapter 126. Display device 124 generally represents any type or form of device capable of visually displaying information forwarded by display adapter 126. Similarly, display adapter 126 generally represents any type or form of device configured to forward graphics, text, and other data for display on display device 124.

As illustrated in FIG. 1, computing system 110 may also include at least one input device 128 coupled to communication infrastructure 112 via an input interface 130. Input device 128 generally represents any type or form of input device capable of providing input, either computer- or human-generated, to computing system 110. Examples of input device 128 include, without limitation, a keyboard, a pointing device, a speech recognition device, or any other input device.

As illustrated in FIG. 1, computing system 110 may also include a primary storage device 132 and a backup storage device 133 coupled to communication infrastructure 112 via a storage interface 134. Storage devices 132 and 133 generally represent any type or form of storage device or medium capable of storing data and/or other computer-readable instructions. For example, storage devices 132 and 133 may be a magnetic disk drive (e.g., a so-called hard drive), a floppy disk drive, a magnetic tape drive, an optical disk drive, a flash drive, or the like. Storage interface 134 generally represents any type or form of interface or device for transferring data between storage devices 132 and 133 and other components of computing system 110.

In one example, databases 140 may be stored in primary storage device 132. Databases 140 may represent portions of a single database or computing device or it may represent multiple databases or computing devices. Alternatively, databases 140 may represent (be stored on) one or more physically separate devices capable of being accessed by a computing device, such as computing system 110 and/or portions of network architecture 200.

Continuing with reference to FIG. 1, storage devices 132 and 133 may be configured to read from and/or write to a removable storage unit configured to store computer software, data, or other computer-readable information. Examples of suitable removable storage units include, without limitation, a floppy disk, a magnetic tape, an optical disk, a flash memory device, or the like. Storage devices 132 and 133 may also include other similar structures or devices for allowing computer software, data, or other computer-readable instructions to be loaded into computing system 110. For example, storage devices 132 and 133 may be configured to read and write software, data, or other computer-readable information. Storage devices 132 and 133 may also be a part of computing system 110 or may be separate devices accessed through other interface systems.

Many other devices or subsystems may be connected to computing system 110. Conversely, all of the components and devices illustrated in FIG. 1 need not be present to practice the embodiments described herein. The devices and subsystems referenced above may also be interconnected in different ways from that shown in FIG. 1. Computing system 110 may also employ any number of software, firmware, and/or hardware configurations. For example, the example embodiments disclosed herein may be encoded as a computer program (also referred to as computer software, software applications, computer-readable instructions, or computer control logic) on a computer-readable medium.

The computer-readable medium containing the computer program may be loaded into computing system 110. All or a portion of the computer program stored on the computer-readable medium may then be stored in system memory 116 and/or various portions of storage devices 132 and 133. When executed by processor 114, a computer program loaded into computing system 110 may cause processor 114 to perform and/or be a means for performing the functions of the example embodiments described and/or illustrated herein. Additionally or alternatively, the example embodiments described and/or illustrated herein may be implemented in firmware and/or hardware.

I. Dynamic Respiratory Classification and Tracking of Pathologies

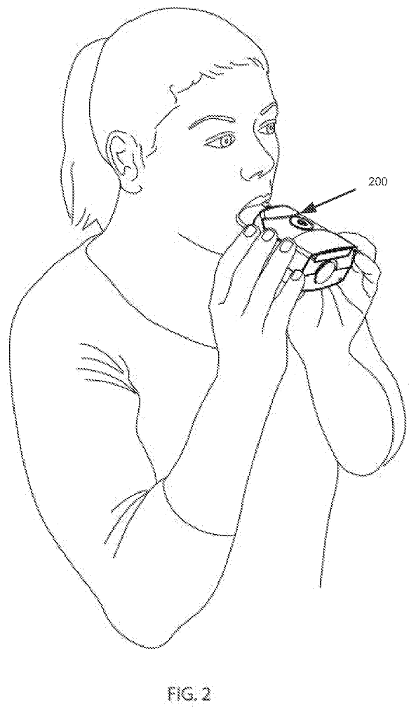

In one embodiment of the present invention, breath sounds are captured by a microphone and flow is captured by a differential pressure sensor. FIG. 2 illustrates a user breathing into an exemplary apparatus comprising a microphone for capturing breathing sounds and a volume sensor for capturing flow signals in accordance with an embodiment of the present invention. As shown in FIG. 2, a user can breathe into device 200. Device 200 will capture both breathing sounds (e.g., the respiratory audio) and can also comprise a volume sensor for capturing breath volume flow signals. To capture the breathing sounds, any sample rate higher than 16 kHz can also adequately be used. A sample rate of 44.1 KHz may be particularly suited.

Breath phase separation can initially be carried out by using a breath flow over time signal and searching for a zero crossing between the lowest peak (inhalation) and the highest peak(exhalation). To make this process more robust, signal that is captured by the microphone and may be present in the audio channel may also be processed and analyzed (as will be explained in more detail later). In one embodiment, the dynamic respiratory classification procedure then classifies the lobes into two different classes that correspond to inhalation and exhalation. This classification can provide timestamps for each inhalation and exhalation event and for rest periods to be able to define a full breath cycle with four phases: inhalation, pause or transition, exhalation, and rest. These timestamps can be collected over several breath cycles.

Embodiments of the present invention may also perform dynamic respiratory classification to diagnose pathologies in patients. Wheezing, a type of respiratory symptom, is a continuous harmonic sound made while breathing and may occur while breathing out (exhalation or cough) or breathing in (inhalation). Wheeze or wheezing sounds occur during breathing when there is obstruction, constriction or restriction in the lung airways and is often indicative of lung disease or heart disease that affects the lungs. Wheeze can be categorized as a whistling sound, a stridor (a high pitched harsh wheeze sound) or rhonchi, (a low pitched wheeze sound). Asthma and chronic obstructive pulmonary disease (COPD) are the most common cause of wheeze. Other causes of wheeze can include allergy, pneumonia, cystic fibrosis, lung cancer, congestive heart failure and anaphylaxis.

It is apparent that the occurrence of wheeze is a diagnostic marker for lung disease and is most commonly detected by listening to the lungs with a stethoscope. Some wheeze sounds may also be heard by the person generating the wheeze or a person nearby, and thus the occurrence of wheeze can also be a patient-reported symptom.

Most people suffering from wheeze-related symptoms have many different types of wheezes, each coming from a narrowed area in the lungs that produces frequencies simultaneously or in a sequence. The frequencies, intensities, behavior and characteristics of wheeze sounds reflect the degree of airway narrowing and the condition of the resonating airway tissue. But, unfortunately, most of it remains hidden or inaudible to the human ear. Digital devices exist that can report the occurrence of wheeze sounds, but these devices will often miss wheeze particles and other characteristics, which may be hidden or inaudible, and yet reflective of lung disease.

Crackles are discontinuous, explosive, unmelodious sounds that are caused by fluid in the airways or the popping open of collapsed airway tissue. They can occur on inhalation or exhalation. Crackles also known as rales, are often categorized as fine (soft and high pitched), medium or coarse (louder and lower in pitch), and can be caused by stiffness, infection, or collapse of the lung airways. They can also be referred to as rattling sounds. Diseases where crackles are common are pulmonary fibrosis and acute bronchitis. Crackles are most commonly heard with a stethoscope, however the number of popping sounds (including their velocity, duration, pitch and intensity) is difficult to hear with the human ear.

Embodiments of the present invention provide an apparatus for evaluating lung pathology that may comprise a microphone or a device with a microphone such as mobile phone that includes a headset and a speaker. The apparatus may comprise one or more of the following devices for lung testing, monitoring and therapy: a mobile phone, a headset, a speaker, a Continuous Positive Airway Pressure (CPAP), an Oscillating Positive Expiratory Pressure Device (OPEP), a spirometer, a stethoscope, a ventilator, cardiopulmonary equipment, an inhaler, an oxygen delivery device and a biometric patch.

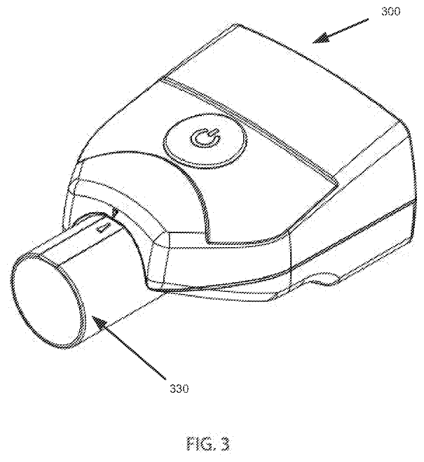

FIG. 3 illustrates an exemplary apparatus 300 comprising a microphone for capturing breathing sounds and a volume sensor for capturing flow signals in accordance with the methods and apparatus of embodiments of the present invention. The apparatus for evaluating lung pathology may be similar to the apparatus illustrated in FIG. 3, used in the methods and apparatus of the present invention. A user breathes into the opening 330 of the device. In one embodiment, apparatus 300 may comprise a conventional microphone, which can be used to record the breathing patterns of the user. By using either a conventional or a device-specific microphone and the accompanying software, the embodiments of the present invention can detect wheeze and crackle related events. Further, device 300 may also comprise a volume sensor that can also be used to detect breath flow signals. Moreover, the test can be self-administered without requiring special testing equipment or trained personnel.

In a different embodiment, the apparatus for evaluating lung pathology may comprise both a microphone for recording breathing sounds and a flow sensor or spirometer that measures the flow and volume of air a subject is contemporaneously breathing in and out. In one embodiment, a single device records both the breathing sounds and measures the breath volume flow.

In one embodiment, a single dual sensor spirometer device may be configured to capture both the breathing sounds and also the breath volume and flow (as will be discussed further later). In one embodiment, the breathing sounds and the breath volume and flow may be advantageously analyzed by the accompanying software in the electronic device (such as an iPad® or iPhone®) to provide a diagnostician an understanding of lung pathologies from which a patient may suffer.

In one embodiment, the apparatus captures respiratory sounds, and sends the respiratory recording to a computing device, which performs dynamic respiratory classification and tracking. The computing device stores the recording and the data in a computerized medium. Embodiments of the present invention provide a significant improvement over conventional methods of detecting wheeze and crackle, because as noted above, while digital devices exist that can report the occurrence of wheeze sounds, this approach will often miss (e.g., fail to detect) wheeze particles and characteristics that are hidden or inaudible and yet reflective of lung disease. Accordingly, embodiments of the present invention allow wheeze sounds to be detected with a high level of sensitivity. Embodiments of the present invention also do not miss wheeze particles and are sensitive enough to recognize wheeze characteristics that are hidden and inaudible to traditional methods of wheeze detection.

Similarly embodiments of the present invention advantageously allow crackles to be detected whereas prior methods of detecting crackle involved the use of non-computerized methods, e.g., using a stethoscope. Embodiments of the present invention comprise a significant improvement to computer related technology by providing hardware and software that is able to detect wheeze sounds and crackles with a high degree of sensitivity.

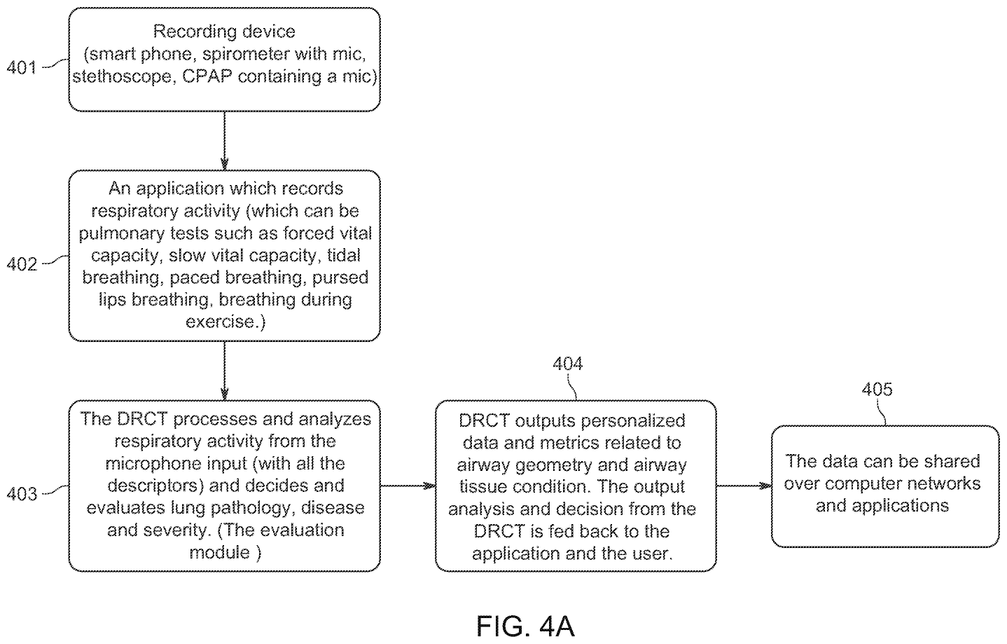

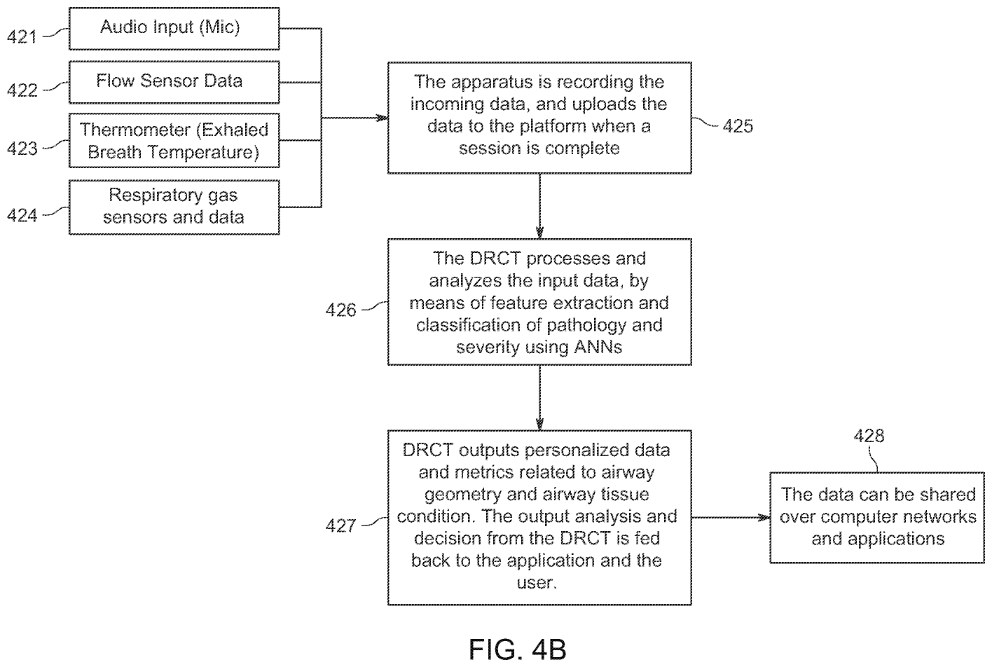

FIG. 4A illustrates an exemplary flow diagram indicating the manner in which a dynamic respiratory classifier and tracker (DRCT) framework can be used in evaluating lung pathology in accordance with an embodiment of the present invention. An example of a DRCT framework is described in U.S. patent application Ser. No. 16/197,025, entitled “METHOD AND APPARATUS FOR TRAINING AND EVALUATING ARTIFICIAL NEURAL NETWORKS USED TO DETERMINE LUNG PATHOLOGY”, filed on Nov. 20, 2018, and which is hereby incorporated by reference in its entirety for all purposes.

At block 401, a recording device (e.g. microphone or a spirometer with a recording device) is used to record breathing sounds of a subject. The recording device can, for example, be a smart phone, a spirometer with a microphone (as will be discussed further below), a stethoscope, a ventilator, an OPEP device, or a CPAP machine with a microphone. As mentioned above, in one embodiment, a device can be configured to record both breathing sounds and measure air volume and flow, e.g., the volume of air a subject can inhale or exhale, and the rate of inhalation or exhalation.

At block 402, an application associated with the recording device (e.g. software installed on the recording device or an accompanying device such as a smart phone) records the audio signal corresponding to respiratory activity. The respiratory activity can be pulmonary testing and monitoring of forced vital capacity, slow vital capacity, tidal breathing, paced breathing, pursed lips breathing, and breathing during exercise.

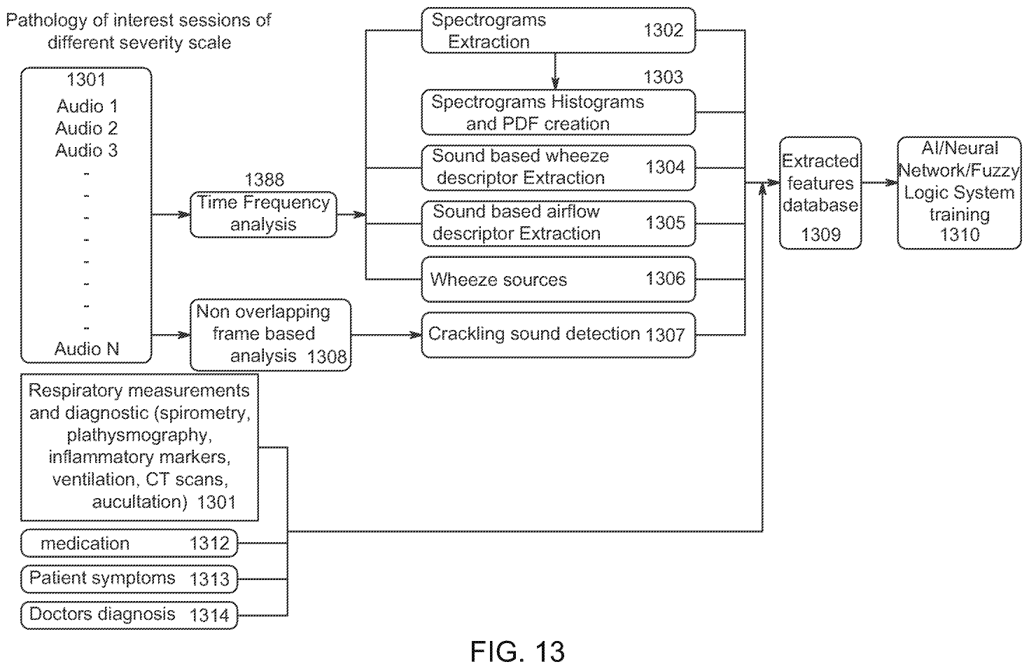

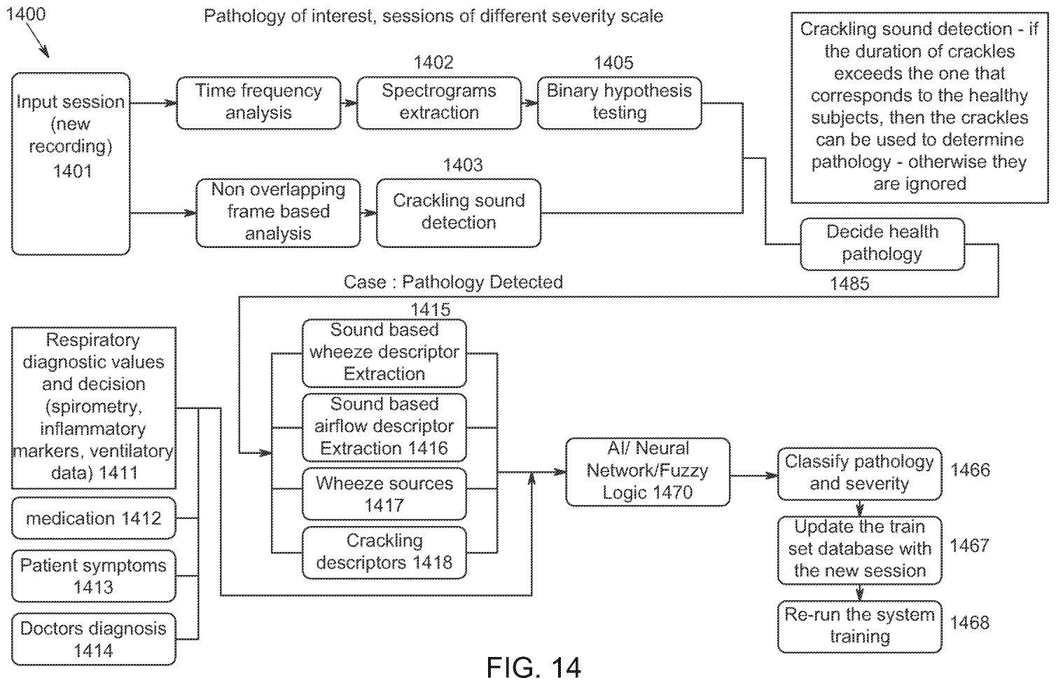

At block 403, the dynamic respiratory classification framework mentioned above (the DRCT framework as described in U.S. patent application Ser. No. 16/197,025, entitled “METHOD AND APPARATUS FOR TRAINING AND EVALUATING ARTIFICIAL NEURAL NETWORKS USED TO DETERMINE LUNG PATHOLOGY”, filed on Nov. 20, 2018, and hereby incorporated by reference in its entirety for all purposes) processes and analyzes respiratory activity from the microphone input. As discussed above, first the breath phases, the breath cycle, and all the descriptors that characterize breathing at rest need to be determined using the DRCT framework. Then the change in the relevant descriptors can be tracked as the patient begins to exercise and increases exercise intensity. The descriptors and the manner in which they change during activity can be used to decide and evaluate lung pathology, disease and severity. Details regarding the manner in which this is done using neural networks will be discussed further in connection with the Training and Evaluation Modules of FIGS. 13 and 14.

At block 404, the DRCT framework outputs personalized data and metrics related to airway geometry and airway tissue condition. The output analysis and decision from the DRCT is fed back to the software application and the user (e.g., software mentioned in connection with FIG. 3).

At block 405, the data can be shared over computer network and with other applications as well.

FIG. 4B illustrates an exemplary flow diagram indicating the manner in which the DRCT framework can be used in evaluating lung pathology where inputs are received from several different types of sensors in accordance with an embodiment of the present invention.

As shown in FIG. 4B, there can be different types of inputs into the DRCT procedure in addition to a microphone (e.g., microphone 421). For example, additional inputs can be received from a flow sensor 422, a spirometer that includes a flow sensor and a volume sensor (not shown in FIG. 4B), a thermometer (to capture exhaled breath temperature) 423, and additional respiratory gas sensors 424.

At block 425, the apparatus recording the incoming data can upload the data to the computer platform (e.g. software discussed in connection with FIG. 3 above) when a session is complete.

At block 426, the DRCT framework processes and analyzes the input data by means of feature extraction and classification of pathology and severity. In one embodiment, the feature extraction and classification is performed using artificial intelligence (AI) processes such as Deep Convolutional Nueral Network (CNN) architectures or other artificial neural networks (ANNs). Subtypes of convolutional neural networks such as Fully Convolutional networks may be particularly suited to the task.

The methodology and system that will be used to classify the recorded data according to disease pathology and severity and is based on artificial neural networks (ANNs). Artificial neural networks are widely used in science and technology. An ANN is a mathematical representation of the human neural architecture, reflecting its “learning” and “generalization” abilities. For this reason, ANNs belong to the field of artificial intelligence. ANNs are widely applied in research because they can model highly non-linear systems in which the relationship among the variables is unknown or very complex. Details regarding the manner in which this is done using neural networks will be discussed further in connection with the Training and Evaluation Modules of FIGS. 13 and 14.

At block 427, the DRCT outputs characteristics and measurements that define a person's individualized airway geometry and morphology including the size and shape of the airways and the condition of the airway tissue. The output analysis and decision from the DRCT is fed back to the application and the user.

At block 428, the data can be shared over computer network and with other applications as well.

As noted above, the apparatus for evaluating lung pathology may also optionally include a spirometer, a ventilator, a flow sensor, a volume sensor, a Continuous Positive Airway Pressure (CPAP) machine, an oscillating positive expiratory pressure device (OPEP), an O2 device and a traditional or digital stethoscope. In one embodiment, signals extracted using these various methods may be synchronized after collection using some distinctive feature of the breath that appears in each signal.

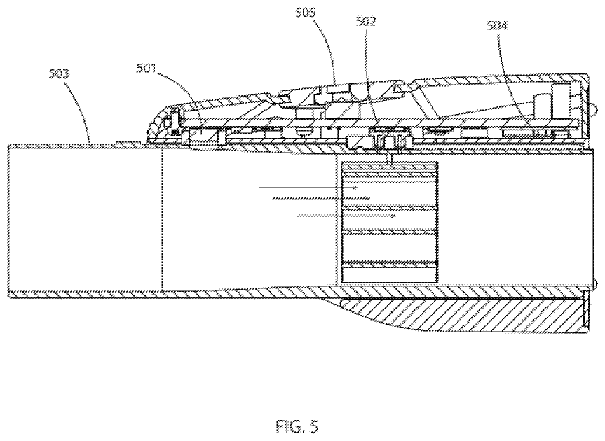

FIG. 5 illustrates a spirometer with built-in lung sound analysis in accordance with an embodiment of the present invention. The spirometer may be a dual or multi-sensor spirometer that comprises a microphone 501, a flow sensor 502 (e.g., a turbine, a differential pressure transducer, an ultrasonic flow measurement device), an optional volume sensor (not shown), a disposable mouthpiece 503, a Bluetooth controller 504, and a battery indicator 505. The entire flow tube may act as a disposable mouthpiece. In one embodiment, the spirometer (a device with a flow sensor and/or volume sensor) comprises an added acoustic sensor or microphone 501. In one embodiment, the flow sensor 502 and the volume sensor may be part of the same unit. The spirometer is a medical measurement device into which a patient breathes. It contains a flow sensor which measures respiratory activity and lung volumes in volumetric units. In other words, the flow sensor measures airflow volume and the speed of airflow in and out of the lungs to detect airflow limitation. In one embodiment, the flow sensor may measure both air flow and lung volume (without needing a separate volume sensor). Conventional spirometers are not sensitive enough for precise diagnostics and tracking. For example, a certain percentage of people with lung disease have normal spirometry test results. Respiratory disease is heterogeneous in nature and can include both airflow limitations and lung sounds such as wheeze and crackles. Conventional spirometers, for instance, may only comprise a flow sensor (which may work to detect airflow limitation but not to recognize lung sounds such as wheeze and crackles). The flow sensor is used to measure lung volume and speed in liters per second. These measurements are used to diagnose and track lung disease, especially asthma and COPD. The problem with these measurements is that when used alone they may be too general for early detection and to predict exacerbations. Patients with lung disease or lung disease progression will get overlooked. It may also be difficult to use spirometry to differentiate asthma from COPD and to be correctly assess the severity. A further disadvantage of spirometry is that results obtained are very dependent on user motivation. Embodiments of the present invention, as will be further explained below, use the respiratory audio in combination with flow and volume measurements to classify pathological conditions and corresponding severity.

Further, another challenge associated with using spirometry alone is that spirometry by itself may not be able to identify disease early, predict exacerbations, or differentiate one lung disease from another. Auscultation of the lungs for bronchial sounds such as wheeze and crackles has been used for centuries as a valuable tool for diagnosis and tracking disease, but is dependent on a doctor listening through a stethoscope or a patient reporting wheeze as a symptom. In both cases, the detection of lungs sounds will be limited to what a doctor and patient can hear.

Embodiments of the present invention add lung sound analysis to improve the sensitivity, and diagnostic and disease tracking capabilities. In other words, embodiments of the present invention add lung sound analysis to spirometry to improve diagnostic and disease tracking capabilities. The lung sound analysis (e.g., using the DRCT framework) is added to the spirometers to provide additional diagnostic data. When a patient, for example, blows into the mouth piece, the maximum force or lung power is a sum of all of the airways as a single stream of air hits the flow sensor. Sound, however, reverberates as the air hits the airway walls. When there is obstruction, narrowing, inflammation or fluid present, it affects the pitch and characteristics of the sound. Accordingly, by adding sound analysis, embodiments of the present invention provide additional data points that can be analyzed to determine lung pathology. For example, the total amount of wheeze and the size and quality of the affected airways can be determined.

In one embodiment, the spirometer device simultaneously records airflow volumes and lung sounds. Standardized measurements of spirometry are combined with the dynamic classification of lung sounds, such as wheeze and crackles (from the DRCT framework), to improve the detection of the presence, progression and severity of lung pathology and disease.

In one embodiment, the spirometer can be connected to mobile devices or personal computers through a physical interface or by using a wireless transmission, e.g. Bluetooth. The power and recording controls may be placed physically on the device (using a digital signal processor, for example, embedded into the device) or may be located on the computer (or smart phone, tablet, laptop, etc.) that is linked to and may control the device. In one embodiment, the data can also be automatically or manually uploaded and stored on a computer or other device. In one embodiment, the feature extraction and classification (related to the DRCT framework) are performed on a processor within the spirometer itself. In a different embodiment, the feature extraction and classification are performed on the computer that is connected to and controls the spirometer. For example, the spirometer may be connected to and may be controlled by a computer executing an application that performs feature extraction and classification of the lung sounds.

In one embodiment, the spirometer comprises a noise suppression module—the noise suppression module may have an additional microphone that may be used for recording and subtracting ambient noise. As mentioned above, conventional spirometers are not sensitive enough for precise diagnostics and tracking Embodiments of the present invention provide spirometers with higher sensitivity—one way for increasing the sensitivity is to equip the spirometers with noise suppression modules and sound analysis capabilities.

In one embodiment, there is a mouthpiece that may fit onto the microphone of a mobile phone or device with a microphone to accurately capture respiratory sounds. Embodiments of the present invention are advantageous because, in comparison with conventional methods, they also use acoustics to detect the presence, progression and severity of lung pathology and disease.

Embodiments of the present invention advantageously extract sound-based wheeze descriptors, spectrograms, spectral profiles, sound-based airflow descriptors and sound based crackle descriptors, all of which can detect and track both the audible and inaudible characteristics of wheezing and crackles that occur in breathing. Ronchi stridor and rub may also be detected.

In one embodiment, as discussed in connection with FIG. 4B, the descriptors are fed into a machine learning system (e.g., Deep CNN, or other types of ANNs) that classifies a respiratory recording as healthy or unhealthy. Further, it determines the type of pathology, the disease and the severity (mild, moderate, severe). The classification may include number and letter severity designations per the Global initiative for Obstructive Lung Disease (GOLD) strategy document for the diagnosis management and prevention of COPD. Examples of lung pathology can include infection, inflammation, and fluid. Examples of lung disease can include asthma, chronic obstructive pulmonary disease (COPD), pneumonia, whooping cough, and lung cancer. Examples of severity can include mild, moderate and severe. In addition, the machine learning system, according to embodiments of the present invention, compares respiratory recordings from the same individual to classify the onset, stability or progression of a lung pathology or disease over time.

II. A. Wheeze Descriptor Extraction Based on Sound Analysis

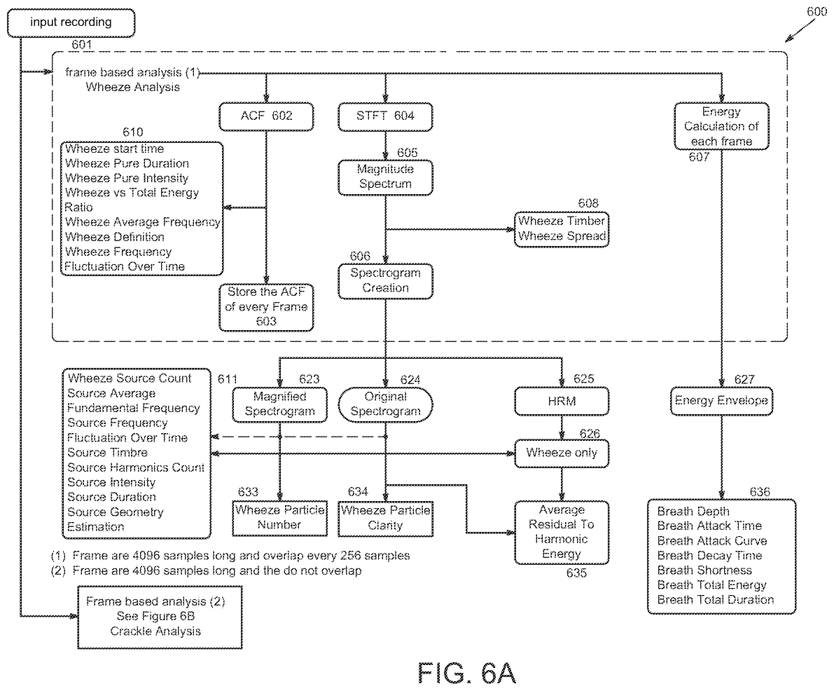

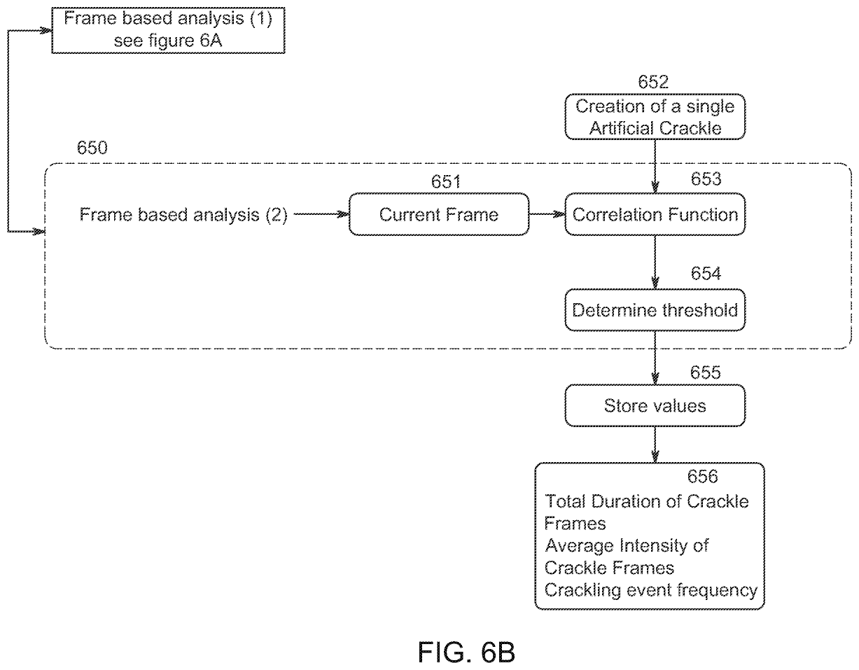

FIG. 6A illustrates a data flow diagram of a process that can be implemented to extract spectrograms and sound-based descriptors pertaining to wheeze in accordance with an embodiment of the present invention. A spectrogram is a time-varying spectral representation that shows how the spectral density of a signal varies with time (it may also be known as a waterfall display).

A wheeze source is defined as a narrowed airway. When turbulent air hits the walls of a narrowed airway, sounds are produced that feature a fundamental frequency and its higher harmonics (or overtones). The spectrogram segments that correspond to these frequencies are called particles.

It should be noted that the spectrogram analysis illustrated in FIG. 6A allows the software (running on the device or computer connected to the microphone or spirometer) to zoom in on the contents and behavior of a single wheeze or more than one wheeze. The spectrogram analysis also enables embodiments of the present invention to advantageously identify wheeze particles (fundamental frequencies and overtones that exist within a single wheeze but are not distinguishable by the human ear).

In the method discussed in connection with FIG. 6A (using spectrograms), embodiments of the present invention use spectrograms that comprise consecutive spectrums (e.g., 10 ms) that are produced using the Fast Fourier Transform (FFT)—this shows the output of the respiratory airways in terms of distribution of energy over frequency over time. This is typically considered a 3 dimensional approach and allows for a higher resolution than a 2 dimensional approach. In particular, it allows the software to zoom in on the contents and the behavior of the wheeze at a granular level.

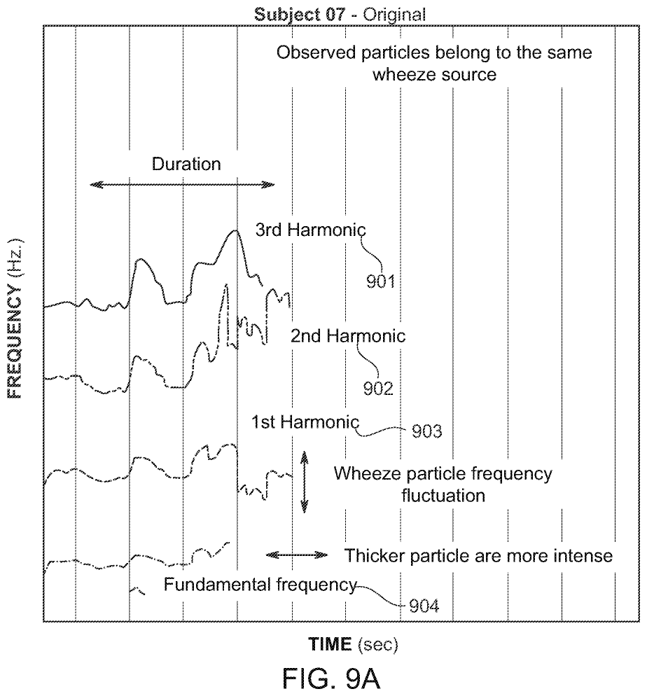

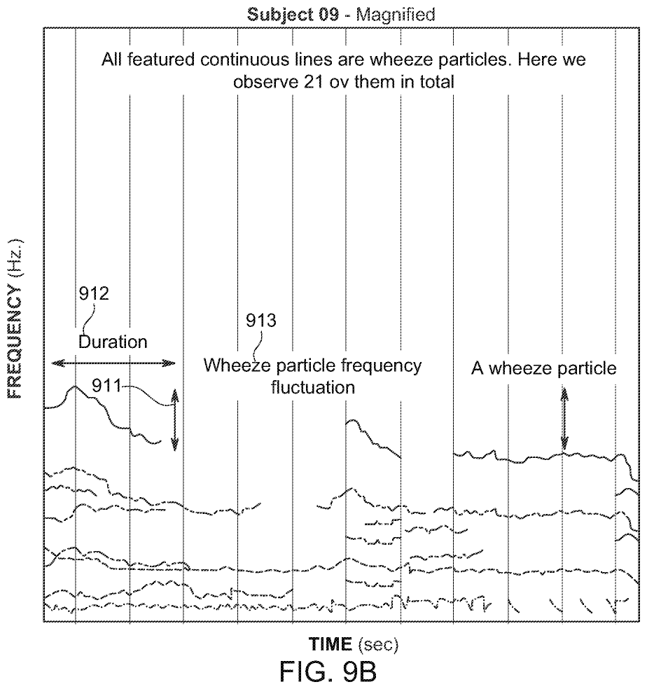

FIG. 9A is an exemplary spectrogram associated with the wheezing behavior of a hypothetical subject in accordance with an embodiment of the present invention. For example, FIG. 9A is an exemplary spectrogram associated with subject “07.’ Each wheeze particle shown in FIG. 9A (namely 901, 902, and 903) belongs to the same wheeze source and is a harmonic of the same source. Each of the wheeze particles has a separate frequency band (or harmonic). In other words, all the three harmonics shown in FIG. 9A (901, 902 and 903) belong to and are extracted from the same wheeze source. The fundamental frequency 904 of the wheeze is represented by a waveform—the thicker line represents more intense wheezing behavior.

As detailed earlier, sound based descriptors from the wheeze signal are extracted by first defining an area of interest. An area of interest can be a breath phase (inhalation, exhalation, cough), a breath cycle or more than one breath phases or breath cycles.

For wheeze analysis, each area of interest is analyzed using overlapping frames. Each frame is 4096 samples long and the overlap is 93% of their duration (every 256 samples). For example, if the sample rate is 44.100 Hz, each frame lasts 92 msecs and the frames overlap every 5 msecs. The values were chosen as such, in order to provide the most temporal and frequency accuracy. It should be noted however that each frame can have a varying number of samples and the overlap duration may also vary.

Referring to FIG. 6A, the sound recording 601 from the patient is received into the wheeze analysis module 600. For each frame, an Auto-Correlation Function (ACF) is determined at block 602. The ACF of every frame is stored at block 603. At block 610, several descriptors can be determined using the ACF values (without needing the spectrogram that is determined at block 605), e.g., wheeze start time, wheeze pure duration, wheeze pure intensity, wheeze vs. total energy ratio, wheeze vs. total duration ratio, wheeze average frequency, wheeze frequency, wheeze definition and wheeze frequency fluctuation over time can be collected and determined.

It should be noted that all the descriptors extracted at blocks 608, 610, 611, 633, 634, 635 and 636 are independent of one another and can be extracted at the same time. Note that wheezing can be identified with the ACF values calculated for each block or frame.

Wheeze Start Time

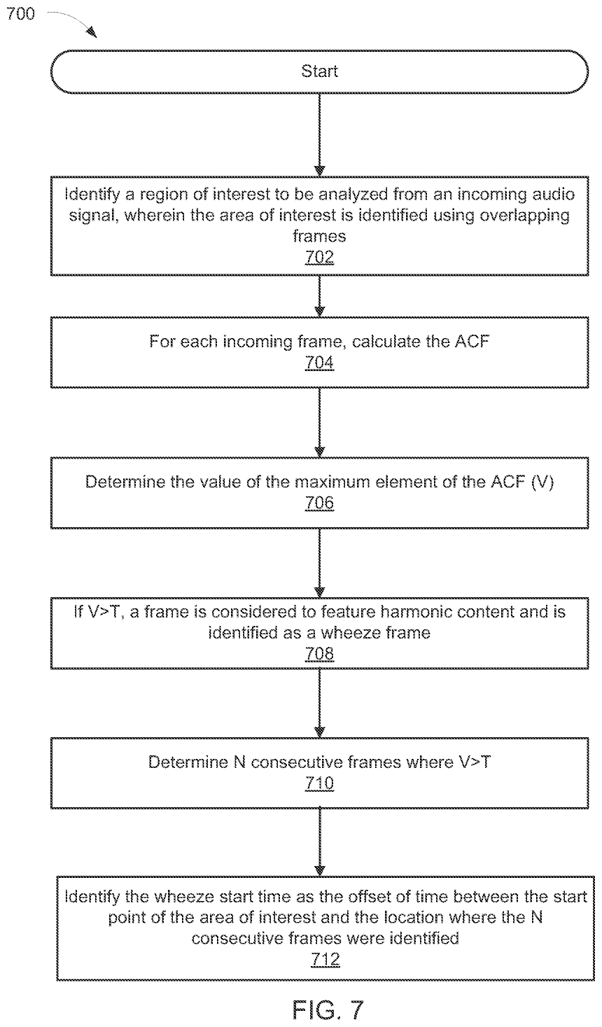

FIG. 7 depicts a flowchart 700 illustrating an exemplary computer-implemented process for detecting the wheeze start time in accordance with one embodiment of the present invention. While the various steps in this flowchart are presented and described sequentially, one of ordinary skill will appreciate that some or all of the steps can be executed in different orders and some or all of the steps can be executed in parallel. Further, in one or more embodiments of the invention, one or more of the steps described below can be omitted, repeated, and/or performed in a different order. Accordingly, the specific arrangement of steps shown in FIG. 7 should not be construed as limiting the scope of the invention. Rather, it will be apparent to persons skilled in the relevant art(s) from the teachings provided herein that other functional flows are within the scope and spirit of the present invention. Flowchart 700 may be described with continued reference to exemplary embodiments described above, though the method is not limited to those embodiments.

At step 702, as noted above, an area or block of interest from the audio signal is identified. Each area of interest is analyzed using overlapping frames. Each frame is 4096 samples long and the overlap is 93% of their duration (every 256 samples). The sample rate is 44.100 Hz which means that each frame lasts 92 msecs and the frames overlap every 5 msecs. As noted above, the frames are not limited to being 4096 samples and similarly the overlap duration is also not limited.

At step 704, for every incoming frame, the software calculates the autocorrelation function (ACF). In one embodiment, the ACF calculations are normalized to the first value so that the maximum value is 1.0. Further, the frequency range of the ACF values can be restricted to be between 100 Hz and 1 KHz.

At step 706, the value of the maximum element of the ACF is determined for each frame.

At step 708 of FIG. 7, the maximum value determined for the frame (V) is compared with a predetermined threshold value (T). In other words T is a predetermined threshold value. If the maximum ACF value determined for the frame is greater than T (V>T), the frame is considered to feature harmonic content and it is designated as a wheeze frame. In one embodiment, T is determined empirically and can be between a range of 0.3 to 0.5—if T falls between the range then the frame is considered to feature harmonic content.

At step 710, if more than N consecutive frames share the property of V>T (where N is the number of frames such that their accumulated duration is greater than 5 milliseconds), the N frames are identified as the start of wheezing.

At step 712, the offset of time between where the area of interest (identified at step 702) started and where the N consecutive frames were identified is designated as the Wheeze Start Time.

As noted above, besides Wheeze Start Time, at step 710, several other descriptors can also be determined using the ACF values, e.g., wheeze pure duration, wheeze pure intensity, wheeze vs. total energy ratio, wheeze vs. total duration ratio, wheeze average frequency, wheeze frequency, wheeze definition and wheeze frequency fluctuation over time. These parameters that may also be determined at block 710 will be discussed below.

Wheeze Pure Duration

The summation of the duration of all the events that are counted as wheeze events, based on the criteria mentioned above, results in the total Wheeze Pure Duration.

Wheeze Pure Intensity

The summation of the intensity of all the frames that have been identified as wheeze frames as described above determines the Wheeze Pure Intensity.

Wheeze Vs. Total Duration Ratio

This descriptor is the ratio of the accumulated duration of all the frames considered as wheeze to the total duration of the Area of Interest.

Wheeze Vs. Total Energy Ratio

To calculate the Wheeze vs. Total Energy Ratio, the software summarizes the energy of the frames accepted as wheeze frames and divides it by the total energy of the Area of Interest. The energy of each frame is calculated as follows:

$$E = \frac{1}{N} {\sum }_{i = 0}^{N} {x}_{i}^{2}$$

where N is the frame length (4096 samples) and x is each sample in the frame.

Wheeze Average Frequency

To calculate the average frequency, the frequency of each particle is calculated. The frequency of the particle can be calculated by determining the position of the ACF where its maximum value is located.

The particle's frequency is defined as

$${f}_{0} = \frac{fs}{N}$$

where f₀ is the wheeze particle's most prominent frequency and fₛ the sample rate of the audio recording.

The average wheeze frequency is given by the following formula:

$${f}_{a v g} = \frac{1}{N} {\sum }_{i = 0}^{N} {f}_{i} .$$

Wheeze Definition

The Wheeze Definition is measured by using the maximum value of the ACF of each wheeze frame. High values indicate that the harmonic connected to wheeze pattern is more clear, whereas lower values indicate a less harmonic wheeze pattern. The wheeze definition is defined as the average of the maximum values of the ACF of the wheeze frames.

Wheeze Frequency Fluctuations Over Time

Frequency fluctuation over time is defined as the variance of the frequency of wheeze frames that comprise wheeze particles. This means that the frames should be consecutive without interruptions for more than a predefined duration.

Referring to FIG. 6A, for each incoming frame into module 600, a Short-Time Fourier Transform (STFT) is calculated at block 604. Alternatively, in a different embodiment, a Fast Fourier Transform (FFT) may be determined at block 604.

At block 605, a magnitude spectrum for each frame is determined using the information from the STFT or the FFT. The STFT (or FFT) and the magnitude spectrum are used to create the sound based descriptors and spectrograms (that could not be extracted using only the ACF values). As mentioned above the spectrograms allow the software to zoom in on the contents and behavior of the wheeze, thereby, advantageously improving the functionality of the computing device.

At block 608, the wheeze timbre and wheeze spread descriptors are determined.

Wheeze Timbre

The wheeze timbre is calculated by averaging the spectral centroid of the wheeze frames. The spectral centroid is a measure used in digital signal processing to characterize a spectrum—it indicates where the “center of mass” of the spectrum is located. The spectral centroid of every wheeze frame is given by

$$μ = {\sum }_{i} {x}_{i} \cdot p ( {x}_{i} )$$

where xᵢ is the magnitude of the frequency bin i and p(x) the probability to observe x:

$$p ( x ) = \frac{S ( x )}{{\sum }_{x} S ( x )}$$

where S is the frequency spectrum and x is the bin index.

Wheeze Spread

The wheeze spread is calculated by averaging the spectral spread of the wheeze frames. The spectral spread of every wheeze frame is given by

$${σ}^{2} = {\sum }_{i} ( {x}_{i} - μ ) \cdot p ( {x}_{i} )$$

where xᵢ is the magnitude of the frequency bin i and μ the spectral centroid.

At block 606 of FIG. 6A, the spectrogram is created. At block 623 a magnified spectrogram is created which is used to determine the wheeze particle number descriptor at block 633. A magnified spectrogram is created because it can be used to identify wheeze particles more clearly than the original spectrogram created at block 624.

FIG. 9A, as discussed above, illustrates a signal spectrogram associated with the wheezing behavior of hypothetical subject “07”. FIG. 9B illustrates an exemplary magnified spectrogram associated with the wheezing behavior of a hypothetical subject in accordance with an embodiment of the present invention. For example, FIG. 9B is associated with the wheezing behavior of a hypothetical subject “09.” FIG. 9B is an example of a magnified spectrogram determined at block 623. All the continuous lines shown in FIG. 9B are associated with wheeze particles. In total, FIG. 9B contains information about 21 different wheeze particles—these wheeze particles can easily be identified visually because the spectrogram is magnified (in comparison to the original spectrogram of FIG. 9A). For example, wheeze particle 911 has duration 912 and a frequency fluctuation span 913.

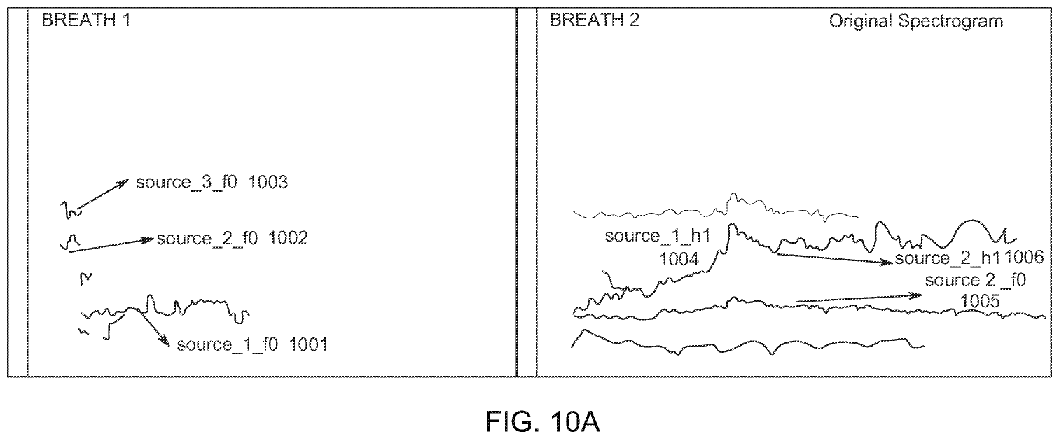

It should be noted that spectrograms illustrated in both FIGS. 9A and 9B are exemplary and have been used for purposes of illustration. FIGS. 10A-10C, by comparison (discussed further below) comprise examples of actual spectrograms extracted from a breathing sound recording of a patient.

Wheeze Particle Number

To calculate the number of wheeze particles, the magnified spectrogram is used where each contributing magnitude spectrum is normalized to each frame's maximum value, making all possible wheeze particles visible. Normalizing to each frame's maximum value magnifies the wheeze particles making each wheeze particle visible.

In one embodiment, an edge detection process may be used (e.g. Sobel with vertical direction), or any other high pass filter operating column-wise on the magnified spectrogram image. The abrupt color changes that happen when wheeze frames occur produce a high value output. This operation is similar to “image equalization.” The spectrograms are treated as images here. Images comprise rows and columns. The normalization is carried out for every column in the spectrogram by dividing the elements of that column with the maximum value of the same column. So, even if the elements of a specific column have small values, when divided by the maximum element, the range of the values for this column is normalized within [0,1] (where 0 is the white color and 1 is the black color). The same process is repeated even if the values within a spectrogram column are high. The result is that all the columns of the spectrogram have the same range [0,1]. This way even particles that are weak in energy show up on the same spectrogram as the high energy ones.

As shown in FIG. 9B, a continuous line is considered a wheeze particle if it crosses over a certain threshold duration. For example, if a continuous line on a magnified spectrogram lasts more than, for example, 5 msecs, the particle count augments by one.

At block 624, the original spectrogram that was created at block 606 is used to determine wheeze particle clarity descriptor at block 634.

Wheeze Particle Clarity

To calculate wheeze particle clarity, the original spectrogram determined at block 606 is used. The result is the accumulation of the output of a high pass filter that processes the spectrogram image column-wise. After the accumulation takes place, the results are divided by the total number of pixels in the spectrogram image. Clear and intense particles usually occurring with more severe wheeze are characterized by a rapid change in color from light to dark. In other words, the wheeze particles associated with more severe pathologies will appear as darker continuous lines on the spectrograms.

FIGS. 10A-10C illustrate the manner in which spectrograms can illustrate wheeze particle clarity in accordance with an embodiment of the present invention.

FIG. 10A illustrates an exemplary spectrogram associated with the wheezing behavior of a hypothetical subject in accordance with an embodiment of the present invention. FIG. 10A comprises spectrograms extracted from two breath cycles, breath 1 and breath 2. Breath 1 comprises three separate wheeze sources, source_1 1001, source 2 1002 and source 3 1003. The fundamental frequency, f0, for each of the wheeze sources is visible on the spectrogram. With respect to breath 2, the first harmonic of wheeze source_1 1004 and the first harmonic of wheeze source_2 1006 are visible. Further, the fundamental frequency of source_2 1005 is also visible on the spectrogram.

As mentioned above, clear and intense particles usually occurring with more severe wheeze are characterized by a rapid change in color from light to dark. As shown in FIG. 10A, during breath 1, source_1 1001 varies in color from light to dark indicating a more severe wheeze. Similarly, during breath 2, source_2 1005 transitions from a lighter color to a darker color also indicating severe wheezing behavior.

FIG. 10B illustrates an exemplary magnified spectrogram which is a magnified version of the spectrogram shown in FIG. 10A in accordance with an embodiment of the present invention. As seen in FIG. 10B, several more wheeze particles are visible because of the magnification. In addition to the wheeze particles that were already visible in FIG. 10A, additional wheeze particles can also be seen in FIG. 10B. For example, the fundamental frequency of source_5 1015, the fundamental frequency of source_6 1016 and the fundamental frequency of source_7 1014 are visible in breath 1 of FIG. 10B. Furthermore, residual airflow sounds 1017 may also be visible on the magnified spectrogram. Similarly, in breath 2, the second harmonic of source_2 1027 is visible (which was not perceptible in the original spectrogram of FIG. 10A).

Another method to determine wheeze particle clarity is the following:

$$WPC = \frac{{\sum }_{i} {\sum }_{j} S ( i , j )}{M \cdot N}$$

where s the spectrogram image, M the image width in pixels, N the image height in pixels, and WPC the wheeze particle clarity.

Average Residual to Harmonic Energy

At block 625 of FIG. 6A, the Harmonic+Residual Model (HRM) is determined.

Subsequently, at block 626, the wheeze-only spectrogram is determined. This is used to determine the Average Residual to Harmonic Energy descriptor at block 635 as will be explained further below. Note that the Average Residual to Harmonic Energy descriptor is the result of the calculation of the HRM.

The HRM is a modeling of the spectrum and, by extension, a modeling of the spectrogram. The modeling process receives a spectrum or spectrogram as an input. The HRM block 625 may receive either the magnified spectrogram at block 623 or the original spectrogram 624 as an input. A peak detection process is employed to detect the locations and the values of the magnitude spectrum peaks. The peaks that are above a threshold (e.g., the threshold can be set at −12 dB) are interpolated with a Blackman-Harris window. The interpolated spectrogram is the harmonic part of the model. In other words, the interpolated spectrogram comprising the harmonic part of the spectrum is the wheeze-only spectrogram. The residual part is obtained by subtracting the interpolated spectrum from the original one. The residual part comprises the residual airflow energies—subtracting out the residual part from the original spectrogram yields the wheeze-only or interpolated spectrogram.

The wheeze-only spectrogram determined at block 626 may be better suited for viewing (and analyzing by the ANN) than the magnitude spectrogram because without the noise added in by the residual airflow energies, the wheeze particles can be clearly viewed on the spectrogram.

FIG. 10C illustrates a wheeze-only spectrogram associated with the wheezing behavior of a hypothetical subject shown in FIG. 10A in accordance with an embodiment of the present invention. As seen in FIG. 10C, with the residual airflow energies filtered out, the wheeze particles can be identified more clearly than in the original or magnified spectrograms of FIGS. 10A and 10B. For example, the wheeze particles for both source_1 1001 and source_2 1005 can be identified more clearly in FIG. 10C as compared to its counterparts FIGS. 10A and 10B.

As noted above, the purpose of the Average Residual to Harmonic Energy descriptor determined at block 635 is to isolate harmonic wheeze sounds and separate them from the simultaneously occurring airflow sounds (or the residual sounds). In other words, the residual refers to the simultaneous airflow sounds that are underneath the wheeze sounds, or occurring at the same time as the wheezing sounds.

To calculate the average residual to harmonic energy, the software extracts an original spectrogram (or magnitude spectrogram), where all of the magnitude spectrum frames are normalized to the maximum intensity value of the entire area of interest.

Using this normalized spectrogram, the software then creates a wheeze-only spectrogram. When a frame is considered to feature harmonic content that is inherent in wheeze sounds, it is normalized and stored into a new spectrogram table. If a frame is not considered as harmonic, then the corresponding table position is filled with zeros.

Subsequently, each magnitude frame that is considered harmonic goes through a peak detection process to detect peaks that lie within the range of (0-12 dB) but at the same time the column-wise Original Spectrum Derivative exceeds a predefined threshold. The locations of these peaks are interpolated with a Blackman-Harris Window that is weighted with the detected peak magnitude value each time.

The resulting spectrogram is then subtracted from the original one, thus the result will not contain the detected wheeze frames (but will contain the residual spectrogram). To calculate the residual airflow energy within the wheeze frames, the software accumulates the values of the residual spectrogram at the indexes that correspond to wheeze frames.

Descriptors Related to Wheeze Source

At block 611, using the wheeze only spectrogram from block 626, several descriptors pertaining to the wheeze source are determined including source duration threshold, maximum number of harmonics, source frequency search range, wheeze source count, source average fundamental frequency, source frequency fluctuation over time, source timbre, source harmonics count, source intensity, source duration, source significance, and source geometry estimation. Each of these descriptors will be discussed further below.

As mentioned earlier, a wheeze source is defined as a narrowed airway. When turbulent air hits the walls of a narrowed airway, sounds are produced that feature a fundamental frequency and its higher harmonics (or overtones). The spectrogram segments that correspond to these frequencies are called particles. The fundamental frequency or pitch of the source is strongly connected to its geometry and how it changes over time. The number and intensity of the harmonics are connected to the force of the airflow and the tissue characteristics of the airway sources. For example, airway tissue that is more firm will produce more harmonics, while airway tissue that is softer and inflamed may produce fewer harmonics. Airways that contain fluid will dampen and reduce the harmonics. For example, as seen in FIG. 9A, the wheeze source comprises a fundamental frequency 904 and three associated harmonics (901, 902 and 903). The wheeze source for the wheeze particles shown in FIG. 9A may be an airway tissue that is firm—accordingly, it produces multiple harmonics.

Sometimes different sources have almost identical frequency characteristics in terms of pitch, number of harmonics and harmonic intensity, thus they overlap. In this case, in one embodiment, the software may define a frequency range around a detected particle of a few hertz that is connected to the first detected particle. This means that there will not be further searching for more particles within this range.

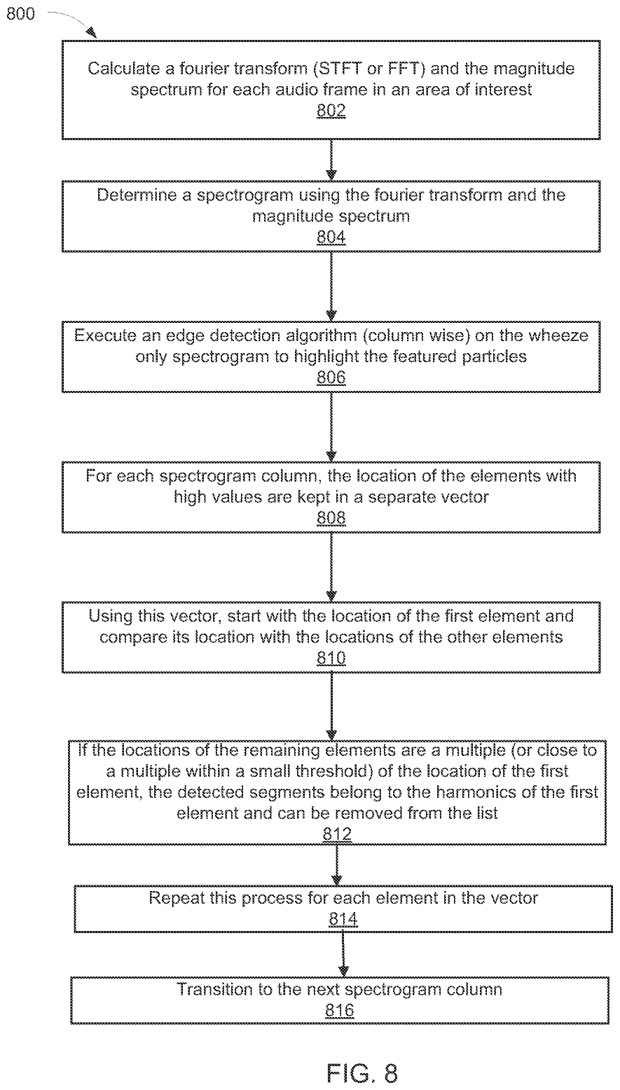

FIG. 8 depicts a flowchart 800 illustrating an exemplary computer-implemented process for determining wheeze source in accordance with one embodiment of the present invention. While the various steps in this flowchart are presented and described sequentially, one of ordinary skill will appreciate that some or all of the steps can be executed in different orders and some or all of the steps can be executed in parallel. Further, in one or more embodiments of the invention, one or more of the steps described below can be omitted, repeated, and/or performed in a different order. Accordingly, the specific arrangement of steps shown in FIG. 8 should not be construed as limiting the scope of the invention. Rather, it will be apparent to persons skilled in the relevant art(s) from the teachings provided herein that other functional flows are within the scope and spirit of the present invention. Flowchart 800 may be described with continued reference to exemplary embodiments described above, though the method is not limited to those embodiments.

At step 802, a STFT or FFT and the magnitude spectrum for each audio frame in an area of interest is determined as indicated above (in connection with blocks 604 and 605 of FIG. 6A).

At step 804, a spectrogram is created (as discussed in connection with block 606 of FIG. 6A).

At step 806, the software executes an edge detection process (column wise) on the spectrogram (e.g., the wheeze only spectrogram created at block 626) to highlight the featured particles.

At step 808, for each spectrogram column, the locations of the elements with high values are stored in a separate vector.

At step 810, using this vector, the software starts with the location of the first element and compares its location with the locations of the remaining ones.

At step 812, if the locations of the remaining elements in the vector are a multiple (or within a small range of the multiple) of the location of the first element, the detected segments belong to the harmonics of the first element, and they are removed from the list.

At step 814, this process is repeated for all the elements in the vector until there are no remaining elements in the vector.

At step 816, the vector is created for the next spectrogram column and the process is repeated.

It should be noted that if the continuity of the lowest in frequency particle breaks before a duration threshold has been reached, nothing gets assigned to that source. In other words, if a particle duration is less than the duration threshold, nothing gets assigned to that source.

As mentioned above, there are several descriptors pertaining to the wheeze source, which are also determined at block 611.

Source duration threshold: The particles associated with the fundamental frequency of a wheeze source should exceed a duration threshold in order to be assigned to a possible source. In one embodiment, this duration threshold is set to 5 milliseconds.

Maximum Number Of Harmonics: In one embodiment, the software can be programmed to search for 5 harmonics per wheeze source (or fewer). In different embodiments, this can be set higher than 5 harmonics.

Source frequency search range: The frequency range of the occurring particles that may be considered as source fundamentals is defined to start at 100 Hz going up to 1 KHz.

Wheeze Source Count: The number of the featured wheeze sources.

Source Average Fundamental Frequency: The average source fundamental frequency. This may also be referred to as the average pitch of the featured sources.

Source Frequency Fluctuation Over Time: The average of the frequency fluctuation over time of a fundamental frequency for each source.

Source Timbre: The source timbre is a measure of the brightness of the source. Each source features a fundamental frequency and a number of harmonics. The location of the fundamental frequency, the number of harmonics and the intensity of the harmonics define the timbre of the source as follows:

$$τ = {\sum }_{k} {\sum }_{i}^{N} {x}_{i} \cdot p ( {x}_{i} )$$

where xᵢ is the magnitude of the frequency bin i and p(x) the probability to observe x:

$$p ( x ) = \frac{S ( x )}{{\sum }_{x} S ( x )}$$

and S(x) represents each column of the wheeze spectrogram.

Source Harmonics Count: This descriptor is related to the average number of harmonics that each source has.

Source Intensity: The average intensity of the featured sources.

Source Duration: The overall duration of the featured sources.

Source Significance: This descriptor is a combination of a few different source characteristics. Specifically, it is the product of the average intensity, duration and pitch.

Source Geometry Estimation: This descriptor provides the dimensions of the resonating wheeze source. This is associated with the source pitch.

Sound Based Airflow Descriptor Extraction

In addition to descriptors pertaining to wheezes, module 600 also determines descriptors pertaining to the airflow recorded as part of the incoming audio recording 601, e.g., at block 636 the software determines breath depth, breath attack time, breath attack curve, breath decay time, breath shortness, breath total energy and breath total duration.

The process to extract the descriptors at block 636 is similar to the other descriptors. For example, the overlapping block based scheme discussed above is used and for every block, the software extracts the associated descriptors.

At block 607, the energy value for each frame is calculated and at block 627 the energy envelope for each frame is determined.

The energy envelope of the input signal is extracted as follows:

For every frame(i), the software calculates

$${e}_{i} = {\sum }_{k} \vert {x}_{k} \vert , i = 0 , 1 , … N ,$$

where xₖ is the kth sample within the frame and e(i) is the energy of the frame.

The descriptors determined at block 636 are as follows:

Breath Area of Interest (A.O.I.) Depth: The value of this descriptor is calculated as follows:

$$B D = \frac{{\sum }_{i} {e}_{x}}{{\sum }_{i} m}$$

where m the maximum value of eₓ and eₓ the envelope of the A.O.I

Breath A.O.I Attack Time: The time in seconds it takes from the A.O.I start until it reaches the 80% of its maximum energy.

Breath A.O.I Attack Curve: The value of this descriptor is calculated as follows:

$$c = \sum \frac{{d}^{2} {e}_{x}}{dx} ,$$

in other words the sum of the second derivative of the envelope of the A.O.I at this stage.

Breath A.O.I Decay Time: The time it takes for the A.O.I to drop down to 10% of the peak of its energy or intensity.

Breath A.O.I Shortness: The time difference Total A.O.I Duration—Decay Time—Attack Time.

Breath A.O.I Total Energy: The total energy of the A.O.I defined as

$$E = \frac{1}{N} {\sum }_{i}^{N} {x}_{i}^{2} .$$

Breath A.O.I Total Duration: The total duration of the A.O.I

II. B. Crackle Descriptor Extraction Based on Sound Analysis

Crackles are impulse like short periodic sounds that repeat rapidly during a defined area of interest. The frequency range of each occurring crackle lies within a range of 100 to 300 Hz. The frames in the frame based analysis pertaining to crackles can be 4096 samples long but they are not required to overlap.

FIG. 6B illustrates a data flow diagram of a process that can be implemented to extract sound based descriptors pertaining to crackling in accordance with an embodiment of the present invention.

When a current frame 651 is received into the crackle module 650, at step 652 a single artificial crackle is created—a filtered impulse response frame is created by filtering a delta function.

$$\begin{matrix}δ ( n ) = 1 & n = 0 \\ δ ( n ) = 0 & n > 0\end{matrix} with a band pass filter with range ( 100 - 300 Hz ) .$$

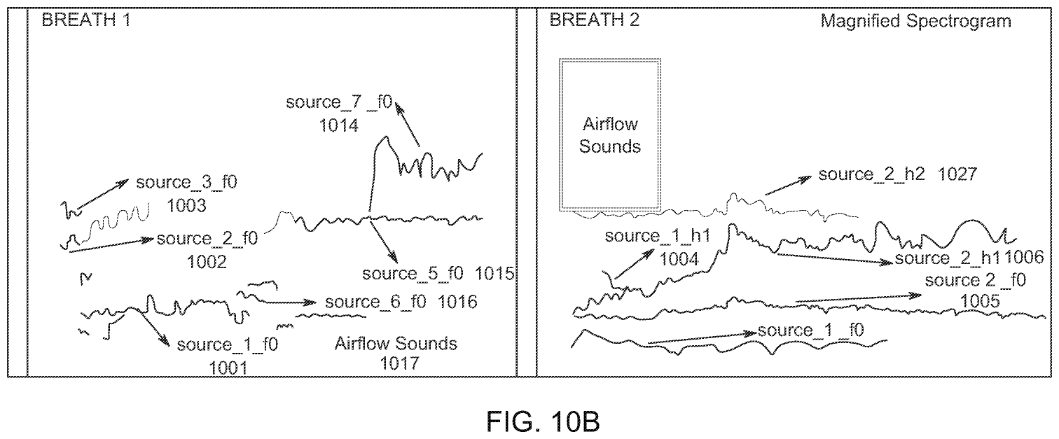

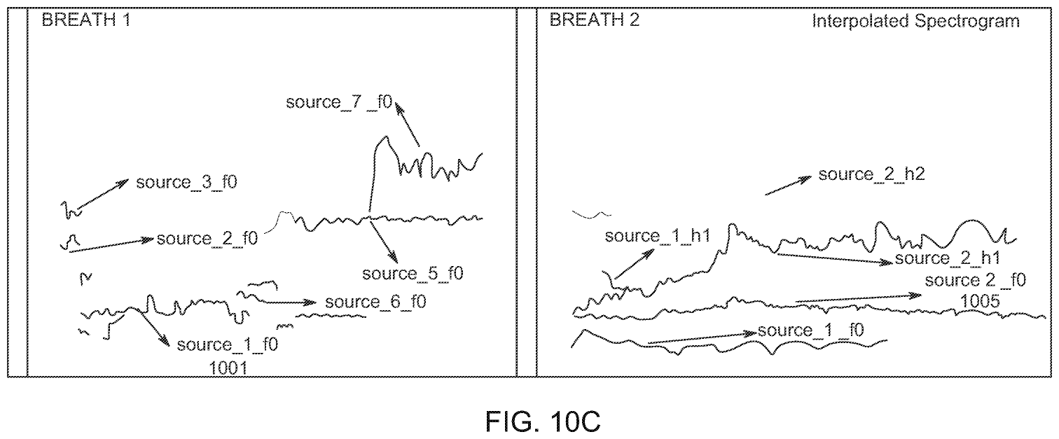

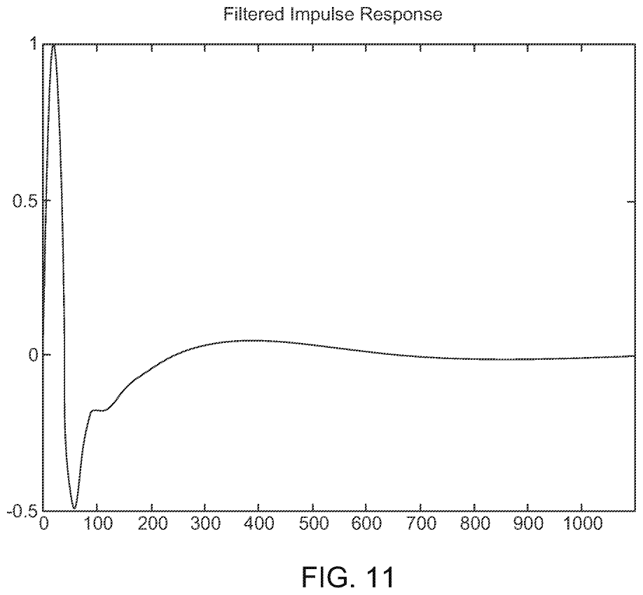

FIG. 11 illustrates the manner in which the filtered impulse response is created by filtering a delta function to create an artificial crackle in accordance with an embodiment of the present invention. The artificial crackle sound is formed by filtering a delta function with a narrow IIR band-pass filter. The filtered frame is the artificial crackle.

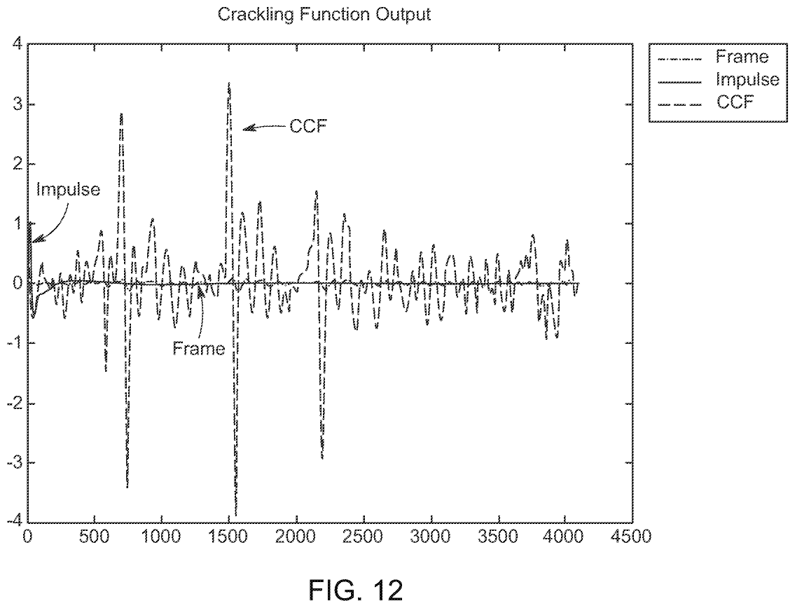

At step 653, a cross correlation function is determined between every frame and the normalized filtered response. FIG. 12 illustrates the cross correlation function determined using the frame and the normalized filtered response in accordance with an embodiment of the present invention. At shown in FIG. 12, the cross correlation function exceeds 1 at certain points—if the cross correlation function exceeds unity at least once, the frame is considered a crackling frame.

Accordingly, at step 654 of FIG. 6B, the thresholds for the cross correlation function (CCF) are determined and, subsequently, at step 655, for every crackling frame, the software stores its time-stamp and its intensity for the feature and descriptor extraction.

At block 656, at least three descriptors pertaining to crackling are determined:

Total duration of crackling frames as the total duration of crackling events.

Average Intensity of crackling frames as the intensity of the frames that feature crackling.

Crackling event frequency as how often crackles happen.

III. Training and Evaluating an Artificial Neural Network (Ann) for Identifying Lung Pathology, Disease and Severity of Disease Using Sound Analysis

In one embodiment of the present invention, an artificial neural network (ANN) can be trained and evaluated to determine lung pathology, disease type and severity. The ANN system for determining lung pathology comprises a training module (shown in FIG. 13) and an evaluation module (shown in FIG. 14). The training and evaluation in this embodiment is performed primarily on the basis of sound based descriptors.

FIG. 13 illustrates a block diagram providing an overview of the manner in which an artificial neural network can be trained to ascertain lung pathologies in accordance with an embodiment of the present invention.

At block 1301 multiple audio files are inputted into the ANN training software, e.g., the audio files may comprise sessions with patients exhibiting symptoms of varying degrees of severity (mild, moderate, severe). Further, the symptoms may relate to a pathology of interest, e.g., asthma.

The audio frames are analyzed both using time frequency analysis (used for analyzing wheezes as discussed above) at block 1388 and using non-overlapping frame based analysis (used for analyzing crackles) at block 1308.

Additionally, the set of respiratory recordings at block 1301 that the training system uses may be annotated by specialists regarding health status, disease, pathology and severity and can include references from other diagnostic tests such auscultation, spirometry, CT scans, blood and sputum inflammatory and genetic markers, etc. The metadata used to annotate the respiratory recordings at block 1301 may comprise respiratory measurements and diagnostics (spirometry, plethysmography, inflammatory markers, ventilation, CT scans, auscultation, etc.), medication 1312, patient symptoms 1313, and doctor's diagnoses 1314.

Other physiological measurements and diagnostics, including pulmonary function testing (spirometry), blood oxygen levels (pulse oximetry), respiratory gas analysis (O2, CO2, VOCs, FeNO), body temperature, and blood and sputum inflammatory and genetic markers can be fed into the ANN processes. In addition, medication usage and tracking, users' symptoms, exercise and diet habits, air quality, and a doctor's diagnosis, can also be fed into the ANN process.

These recordings together with the annotated metadata comprise the “training set.” The ANN processes initially analyze the recordings contained in the training set by employing the frame-based analysis of wheeze module 600 and crackle module 650 in order to tune the ANN processes that will later evaluate new incoming recordings to determine whether they are associated with healthy lungs, and if not, then to determine lung pathology and disease type (e.g., asthma, COPD, etc.) and severity (mild, moderate, severe).

Each recording in the training set is analyzed using overlapping frames (as discussed in connection with wheeze module 600 above) at block 1388. These frames are 4096 samples long and the overlap by 93% of their duration (every 256 samples). For example, if the used sample rate is 44.100 Hz, each frame lasts 92 msecs and the frames overlap every 5 msecs. The exemplary values were chosen to provide temporal and frequency accuracy. It should be noted that both the frame lengths and the overlap duration can vary.

Subsequently, the recordings are used to extract the various descriptors and images discussed above. For example, the spectrogram images are extracted at block 1302. Original spectrograms are created for each respiratory recording. These spectrograms are used to create probability density functions (PDFs) at block 1303. The PDFs that correspond to a specific health status (healthy lungs, mild asthma, moderate asthma, severe asthma, etc.) are averaged.

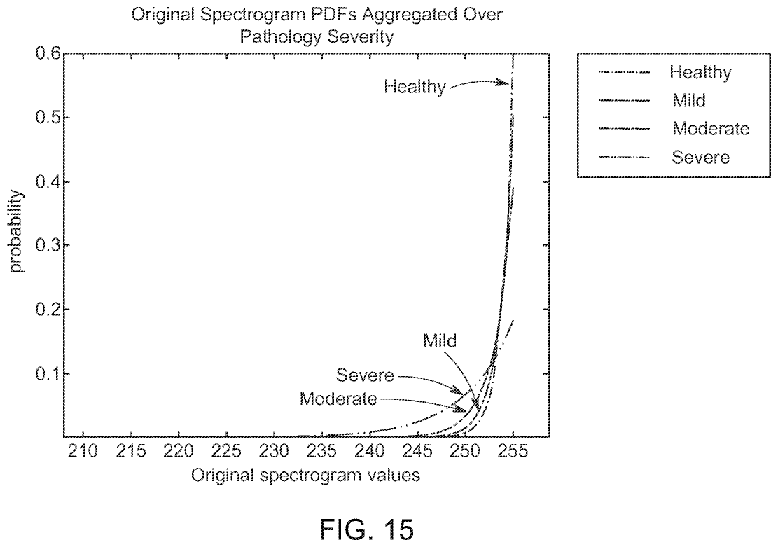

FIG. 15 illustrates exemplary original spectrogram PDFs aggregated over pathology and severity in accordance with an embodiment of the present invention. As will be discussed further below, the PDFs are used in the evaluation module (discussed in connection with FIG. 14) to decide if a new respiratory recording inputted into the ANN belongs to a healthy category or to a category indicating disease by employing a Binary Hypothesis Likelihood Ratio Test.

At block 1304 sound based wheeze descriptors are extracted (e.g. the descriptors extracted at block 610, 633, 634, 608, and 635). At block 1306, wheeze source and the associated descriptors are determined (e.g., descriptors determined at block 611). Additionally, at block 1305, descriptors associated with sound based airflow are extracted (e.g. descriptors extracted at block 636).

Using the non-overlapping frame based analysis at step 1708, the descriptors pertaining to crackle are also extracted at block 1707 (e.g., the descriptors from block 656).

The next step is to store all the extracted spectrograms and descriptor, wherein the values for each of the respiratory recordings are stored separately in the extracted features database at block 1309. The descriptors are also aggregated over pathology and severity to tune the neural network layers and coefficients at block 1310.

FIG. 14 illustrates a block diagram providing an overview of the manner in which an artificial neural network can be used to evaluate a respiratory recording associated with a patient to determine lung pathologies and severity in accordance with an embodiment of the present invention.

The evaluation or decision-making module 1400 shown in FIG. 14 receives as an input a new recording at block 1401. The evaluation module then applies time frequency analysis and extracts a spectrogram (and associated PDF) at block 1402. This is similar to the way in which spectrograms and PDFs are extracted at blocks 1302 and 1303 in the training process shown in FIG. 13. Further, at block 1402, a histogram of the extracted spectrogram (either original spectrogram or a magnified spectrogram) is calculated. This histogram can be used to obtain the session's PDF.

The PDF can be obtained as follows:

$${P}_{i} = \frac{{H}_{i}}{{\sum }_{i} {H}_{i}}$$

where: Hᵢ the histogram elements.

The decision-making module also applies non-overlapping frame based analysis and extracts sound descriptors pertaining to crackling at block 1403. Accordingly, the evaluation module analyzed both the wheeze-based spectrograms and descriptors to determine pathology as well as the crackle-based descriptors.

At block 1405, for the wheeze-based analysis, a binary hypothesis test is performed to determine if the recording is associated with a healthy patient or if the patient is showing characteristics of disease or pathology, which may need further investigation. The binary hypothesis test may provide a binary (true/false) response when evaluating a patient's condition. This binary decision can be carried out after the PDFs in the training set are averaged and the resulting PDFs are correlated with a pathology pattern (mild to severe as shown in FIG. 15). The PDF of the session with the new patient during evaluation can then be compared to the averaged PDFs developed during the training session. In other words, the PDF of the new recording from the patient (obtained at block 1401) can be mapped onto the averaged PDFs determined during the training session to determine if there is a match between the PDF from the new session and any of the pathology patterns as determined during the training session.

The Binary Hypothesis Test performed at block 1405 has the following form:

$$Λ = {\sum }_{n = 1}^{N} ϕ ( {x}_{n} ) {{=}_{\begin{matrix}< \\ {H}_{1}\end{matrix}}}^{\begin{matrix}{H}_{0} \\ >\end{matrix}} 0$$

$$where :$$

$$ϕ ( x ) = log ( \frac{{f}_{H} ( x )}{{f}_{A} ( x )} ) {f}_{H} ( x ) : healthy , {f}_{P} ( x ) : pathology PDF ’ s$$

$$Λ > 0 decide healthy$$

$$Λ < 0 decide pathology$$

$$Λ = 0 decide randomly$$

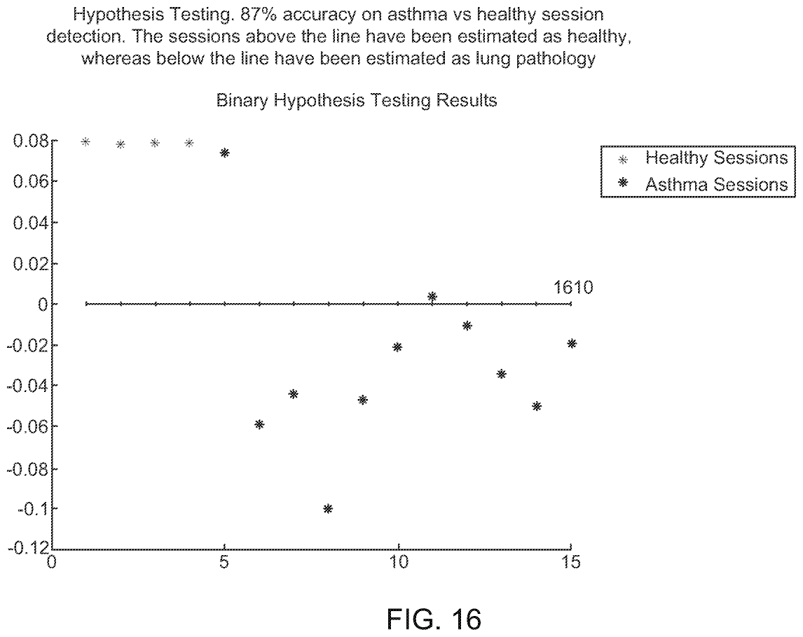

FIG. 16 illustrates exemplary results from the binary hypothesis testing conducted at block 1405 of FIG. 14 in accordance with an embodiment of the present invention. The binary hypothesis testing on incoming new sessions is conducted after the ANN has been trained with a prior data set. As seen in FIG. 16, the sessions associated with points above line 1610 are estimated as healthy, whereas the sessions associated with points below the line 1610 are estimated as related to lung pathology.

Subsequent to the binary hypothesis testing, a recording that has been identified as healthy (or containing no indicia of pathology) may not need to be analyzed further—it is stored as part of the user or patient profile in an associated database for future reference. Each subject's complete data is stored in the database. Each time a new respiratory recording related to the patient is fed into the system, the test is repeated taking into account the stored data in order to detect a possible statistical change that could mean that early stages of pathology or lung disease are present.

In one embodiment, if neither the binary hypothesis testing performed at block 1405 (FIG. 14) and the crackling sound detection at block 1403 show any indications of a pathology (in other words, if both methods of analyzing the new input session or recording from the patient indicate that the patient's lungs are healthy), then the analysis can optionally be stopped at block 1485. In other words, only if a pathology is detected does the analysis progress further. Alternatively, in a different embodiment, the analysis can continue by extracting descriptors at blocks 1415-1418 even if the patient has healthy lungs.

When the respiratory recording is characterized as a pathology at block 1485, the descriptor extraction modules (sound based wheeze descriptors at block 1415, sound based airflow descriptors at block 1416, wheeze source descriptors at block 1417, crackling descriptors at block 1418) are employed to extract the pathology and disease related features. The descriptor extraction modules are similar to the modules 1302, 1303, 1304, 1305, 1306 and 1307 discussed in connection with FIG. 13. The descriptors and all the metadata information from blocks 1411, 1412, 1413 and 1414 are fed into the ANN module 1470. The ANN module 1470 then determines the pathology, disease and severity at block 1466 using the information learned from the processing of the training sets.

As mentioned above, the metadata may include other physiological measurements and diagnostics, including pulmonary function testing (spirometry), blood oxygen levels (pulse oximetry), respiratory gas analysis (O2, CO2, VOCs, FeNO), body temperature, plethsmography, CT scans, and blood and sputum inflammatory and genetic markers can be fed into the ANN processes. Medication usage and tracking, a users' symptoms, exercise and diet, and a doctor's diagnosis, can also be fed into the ANN process. The user's gender, height, weight and race may also be of value.

The session is then classified at block 1466 and is subsequently stored to the training database at block 1467 in order to augment the training set. Subsequently, the process re-runs the training to update its state at block 1468. The extracted features may also be stored to the user profile database in order to compare the new user data to the previous user data for tracking purposes. If a new recording shows characteristics of pathology or disease progression, its characteristics can be compared to the data that has been extracted from older recordings in order to estimate the rate of pathology or disease progression.

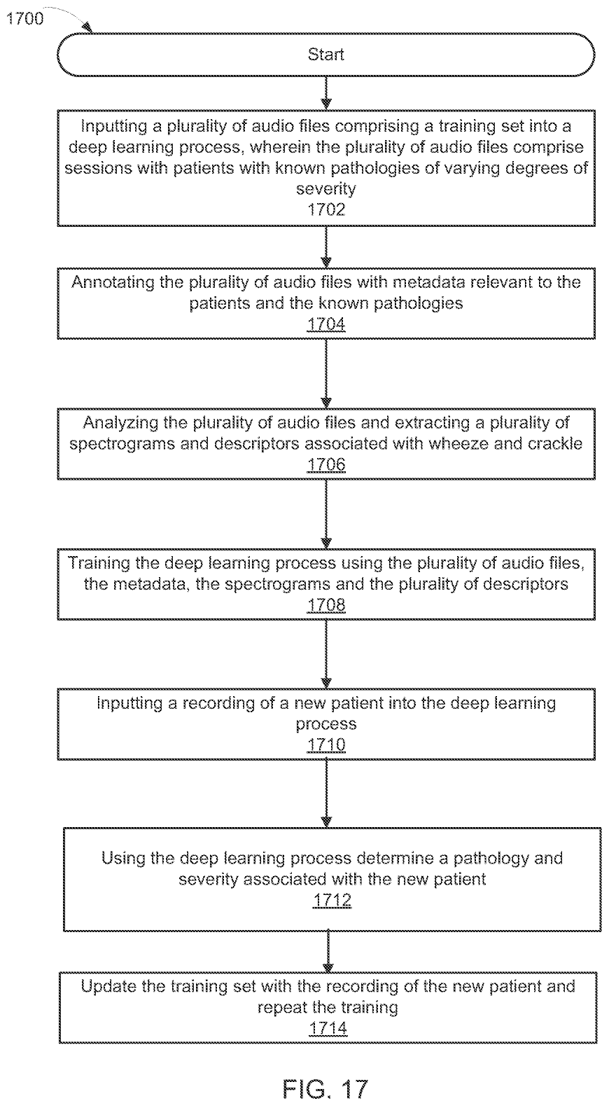

FIG. 17 depicts a flowchart 1700 illustrating an exemplary computer-implemented process for determining lung pathologies and severity from a respiratory recording using an artificial neural network in accordance with one embodiment of the present invention. While the various steps in this flowchart are presented and described sequentially, one of ordinary skill will appreciate that some or all of the steps can be executed in different orders and some or all of the steps can be executed in parallel. Further, in one or more embodiments of the invention, one or more of the steps described below can be omitted, repeated, and/or performed in a different order. Accordingly, the specific arrangement of steps shown in FIG. 17 should not be construed as limiting the scope of the invention. Rather, it will be apparent to persons skilled in the relevant art(s) from the teachings provided herein that other functional flows are within the scope and spirit of the present invention. Flowchart 2700 may be described with continued reference to exemplary embodiments described above, though the method is not limited to those embodiments.

At step 1702, a plurality of audio files comprising a training set are inputted into a computer implemented artificial neural network (ANN) or deep learning process. The plurality of audio files comprise sessions with patients with known pathologies of varying degrees of severity.

At step 1704, the plurality of audio files are annotated with metadata relevant to the patients and the known pathologies. For example, the metadata used to annotate the respiratory recordings at block 1401 may comprise respiratory measurements and diagnostics 1411 (spirometry, plethysmography, inflammatory markers, ventilation, CT scans, auscultation, etc.), medication 1412, patient symptoms 1413, and doctor's diagnoses 1414. Other physiological measurements and diagnostics, including pulmonary function testing (spirometry), blood oxygen levels (pulse oximetry), respiratory gas analysis (O2, CO2, VOCs, FeNO), body temperature, and blood and sputum inflammatory and genetic markers can be fed into the ANN processes. In addition, medication usage and tracking, users' symptoms, exercise and diet habits, air quality, and a doctor's diagnosis, can also be fed into the ANN process. The users gender, height, weight and race may also be of value.

At step 1706, the plurality of audio files are analyzed and a respective spectrogram is extracted for each of the audio files. Further, a plurality of descriptors associated with wheeze and crackle are determined from the plurality of audio files.

At step 1708, the deep learning process is trained using the plurality of audio files, the spectrograms, the descriptors, and the metadata (e.g. as shown at block 1310).

At step 1710, a new recording from a new patient is inputted into the deep learning process. At step 1712, using the deep learning process a pathology is determined with an associated severity for the new patient. As mentioned above, the pathology determination is made using a binary hypothesis testing process. Further, the pathology determination is made using both crackle sound descriptors and analyzing spectrograms for wheeze-related symptoms.

At step 1714, the training set of audio files is updated with the recording of the new patient and the training process is repeated with the additional new recording. Subsequent new recordings are analyzed with the updated deep learning process and the results stored.

IV. Training and Evaluating a Convolutional Neural Network (CNN) for Identifying Lung Pathology, Severity of Disease, and Progression of Disease Over Time Using Sound and Breath Flow/Volume Analysis

As mentioned, typically respiratory analysis systems like the ones described in connection with FIGS. 13 and 14 are capable of recording respiratory audio associated with a patient, analyzing the audio and providing feedback regarding possible pathologies from which the patient may be suffering. Some of these respiratory analysis systems, like the ones discussed in FIG. 13 or 14, use artificial neural networks that are trained to analyze the audio files to diagnose the pathologies. The principal drawback of certain conventional respiratory analysis systems is that only part of the available information (in particular, respiratory sounds) available from the patient is collected, processed, and displayed. Accordingly, conventional respiratory analysis systems are unable to process events and information that would lead to a deeper understanding of the pathology and the manner in which a particular disease or pathology progresses over time.

Embodiments of the present invention use respiratory audio data in conjunction with breath volume and flow data to gain a deeper understanding of a patient's pathology and the manner in which a particular disease or pathology progresses over time. In one embodiment, both audio signals and breath flow are captured by a dual or multi-sense spirometer such as the one discussed in conjunction with FIG. 5.

As discussed above, capturing breath flow in conjunction with audio signals provides distinct advantages. Analysis of breath flow and audio signals collected simultaneously may be used to suppress ambient noise. Audio in the absence of any detected breath flow is likely ambient noise. The ambient noise captured from the audio signal in the absence of any breath flow can be filtered out of the audio signal to improve signal strength and integrity.

More importantly, however, flow and audio signals collected simultaneously allows the spirometer to extract custom features that are descriptive of breathing quality but also of respiratory pathology severity. In other words, flow/volume signals in conjunction with audio signals advantageously provide unique insight into patient pathology and severity that importantly could not be extracted from the respiratory audio signal alone. Furthermore, the combination of the flow/volume signals and audio signals allows descriptor and description combinations to be extracted that were not possible using only the sound-based extraction methods discussed in FIGS. 6A and 6B.

Accordingly, embodiments of the present invention provide an extension of the processes and methods discussed in connection with FIGS. 13 and 14 by accounting for respiratory flow and volume in addition to the audio signals. By using predictors associated with at least one or both of the audio and flow/volume signals, embodiments of the present invention can advantageously predict pathologies earlier in the development cycle and also track pathology severity by regularly analyzing patient sessions. Embodiments of the present invention can use selected descriptors or selected descriptor combinations (derived from either one or both of the flow/volume signals and audio signals) in combination with prediction models, statistical change or machine learning processes to track pathology development and severity.

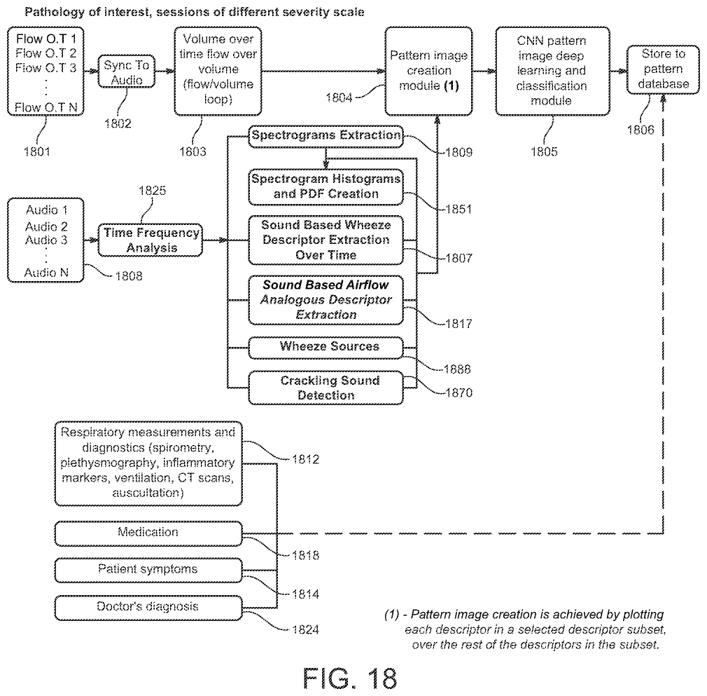

FIG. 18 illustrates a block diagram providing an overview of the manner in which a convolutional neural network (CNN) can be trained to ascertain lung pathologies in accordance with an embodiment of the present invention.

Note that while the Artificial Intelligence (AI) network at block 1310 trained in connection with FIG. 13 typically comprises an Artificial Neural Network (ANN) network, the AI network module 1805 trained in connection with FIG. 18 typically comprises a convolutional neural network (CNN). This is because the ANN at block 1310 of FIG. 13 is trained using the descriptors and spectrograms extracted using the respiratory audio signal. Meanwhile, the CNN module 1805 of FIG. 18 is trained using images developed by plotting certain selected descriptors (associated with either flow/volume signals or respiratory signals) in a subset against other descriptors in the subset as will be discussed further below.

As is well-known, ANN processes input in a different way than CNN. As a result, ANN is sometimes referred to as a Feed-Forward Neural Network because inputs are processed only in a forward-facing direction. Because of the reliance on valid data inputs, ANN tends to be a less popular choice when analyzing images. Meanwhile, CNN works in a compatible way with images as input data. Using filters on an image results in feature maps. CNN doesn't process data in a forward-facing way but rather refers to the same data multiple times when creating maps. Because the CNN pattern image deep learning and classification module 1805 trains using images of selected descriptors in a subset plotted against other descriptors in the subset, it is beneficial for module 1805 to use a CNN rather than an ANN.

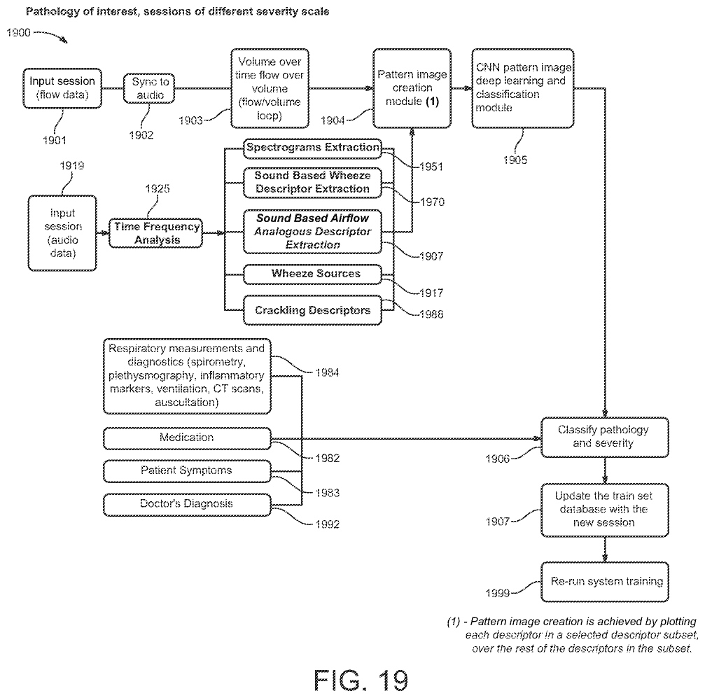

In one embodiment of the present invention, a CNN can be trained and evaluated to determine lung pathology, disease type, early on-set of pathology, severity and trending of the lung pathology over time. The CNN system for determining lung pathology comprises a training module (shown in FIG. 18) and an evaluation module (shown in FIG. 19). Unlike the training and evaluation modules of FIGS. 13 and 14, the training and evaluation in this embodiment is performed on the basis of images created from descriptors derived from both the flow/volume readings and the audio readings retrieved from a dual-sense spirometer (similar to the one discussed in connection with FIG. 5). In one embodiment, the flow sensor and the audio microphone are mechanically and/or electrically coupled together in the same device (e.g., the dual-sense spirometer).

As shown in FIG. 18, at block 1801, breath flow (flow over time) is collected from a sensor, e.g., a sensor incorporated into a spirometer. At block 1802, the breath flow parameters collected from the spirometer at block 1801 are synced with the audio respiratory signals collected at block 1808. Similar to the audio files collected in FIG. 13, the audio files collected at block 1808 may comprise sessions with patients exhibiting symptoms of varying degrees of severity (mild, moderate, severe). Further, the symptoms may relate to a pathology of interest, e.g., asthma. Further, the breath flow readings collected at block 1801 may also comprise sessions with patients exhibiting symptoms of varying degrees of severity (mild, moderate, severe) and relate to a pathology of interest, e.g., asthma.

At block 1825, time frequency analysis is conducted on the audio respiratory signal captured at block 1808. This time frequency analysis is substantially similar to the one conducted by block 1388 in FIG. 13. In one embodiment, the analysis is performed over discrete intervals which may facilitate the graphical representation of the output (which may be inputted into a CNN module 1805). In other words, descriptor values are extracted over time so that a separate vector for each descriptor is generated in order to be able to overlay patterns (e.g., plot the descriptors one over another to obtain the appropriate input image) for the CNN. It comprises spectrogram extraction performed at block 1809, which is substantially similar to the spectrogram extraction performed at block 1302 in FIG. 13. As mentioned above, original spectrograms are created for each respiratory recording. These spectrograms are used to create spectrogram histograms and probability density functions (PDFs) at block 1851 (substantially similar to the manner in which they are created at block 1303 in FIG. 13).

The PDFs that correspond to a specific health status (healthy lungs, mild asthma, moderate asthma, severe asthma, etc.) are averaged. Note that FIG. 15 illustrates exemplary original spectrogram PDFs aggregated over pathology and severity in accordance with an embodiment of the present invention. Further note that the PDFs may be used in the evaluation module (discussed in connection with FIG. 19) to decide if a new respiratory recording that is being evaluated by the AI module belongs to a healthy category or to a category indicating disease by employing a Binary Hypothesis Likelihood Ratio Test (discussed in connection with FIG. 16).

Substantially similar to block 1304 in FIG. 13, at block 1807 of FIG. 18 sound-based wheeze descriptors are extracted (e.g. the wheeze-based descriptors extracted in connection with the discussion for FIG. 6A). At block 1888 wheeze source and the associated descriptors are determined (e.g., descriptors determined at block 611 of FIG. 6A). Additionally, at block 1817 (substantially similar to block 1305 in FIG. 13) sound-based airflow analogous descriptors are extracted (e.g. descriptors extracted at block 636 of FIG. 6A).

In one embodiment, each recording from block 1808 in the audio recording training set is analyzed using overlapping frames (as discussed in connection with wheeze module 600 in FIG. 6A above). These frames are 4096 samples long and the overlap by 93% of their duration (every 256 samples). For example, if the used sample rate is 44.100 Hz, each frame lasts 92 msecs and the frames overlap every 5 msecs. The exemplary values were chosen to provide temporal and frequency accuracy. It should be noted that both the frame lengths and the overlap duration can vary.

Note that the crackling sound detection conducted at block 1870 of FIG. 18 may be based on the time-frequency analysis conducted at block 1825. While the crackling sound detection at module 1307 of FIG. 13 was conducted based on the non-overlapping frames analysis at block 1308, the crackling sound detection analysis conducted at block 1870 of FIG. 18 may be based on a time-frequency analysis.

However, in a different embodiment, the crackling sound detection block 1870 may be conducted based on a non-overlapping frame analysis as previously discussed in connection with module 1307 of FIG. 13. In such an embodiment, the audio frames could be analyzed both using time frequency analysis (used for analyzing wheezes as discussed above in connection with FIG. 13) and also using non-overlapping frame based analysis (used for analyzing crackles).

Additionally, at block 1812, the set of respiratory recordings that the training system uses may be annotated by specialists regarding health status, disease, pathology and severity and can include references from other diagnostic tests such auscultation, spirometry, CT scans, plethysmography, ventilation, blood and sputum inflammatory and genetic markers, etc. The metadata used to annotate the respiratory recordings at block 1812 may comprise respiratory measurements and diagnostics at block 1812 (spirometry, plethysmography, inflammatory markers, ventilation, CT scans, auscultation, etc.), medication 1818, patient symptoms 1814, and doctor's diagnoses 1824, among other things. The metadata collected at blocks 1812, 1818, 1814 and 1824 may be inputted into the pattern database at block 1806, which stores the metadata along with the classified pattern images from the CNN pattern image deep learning and classification module 1805.

In one embodiment, the patient information data and metadata collected at blocks 1812, 1818, 1814 and 1824 may be used to annotate the descriptors and spectrogram extracted at blocks 1809, 1851, 1807, 1817, 1888 and 1870 prior to feeding the information to the pattern image creation module 1804. In other words, the patient information data and metadata may be part of the training set that is processed by the CNN module 1805.

Other physiological measurements and diagnostics, including pulmonary function testing (spirometry), blood oxygen levels (pulse oximetry), respiratory gas analysis (O2, CO2, VOCs, FeNO), body temperature, and blood and sputum inflammatory and genetic markers can also be stored in the database at block 1806 and used for evaluation (as will be discussed in connection with FIG. 19). In addition, medication usage and tracking, users' symptoms, exercise and diet habits, air quality, and a doctor's diagnosis, can also be stored.

As mentioned above, at block 1808, the flow data collected by the spirometer at block 1801 is synchronized to the audio recordings collected at block 1808. In other words, the respiratory audio and the descriptors extracted therefrom can be synchronized to the respiratory flow and the descriptors extracted therefrom, e.g., the volume over time, the flow over volume (the flow/volume loop graph). In one embodiment, the audio and flow data can be collected and consolidated in a way such that they are inherently synchronized. At block 1803, using the flow information received from block 1801, the volume over time, the flow over volume (the flow/volume loop) are determined.

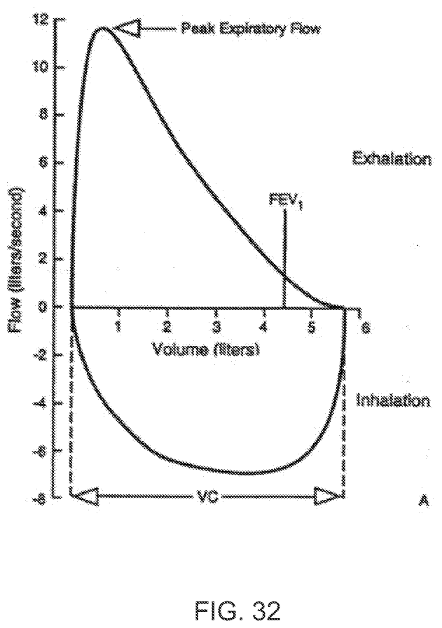

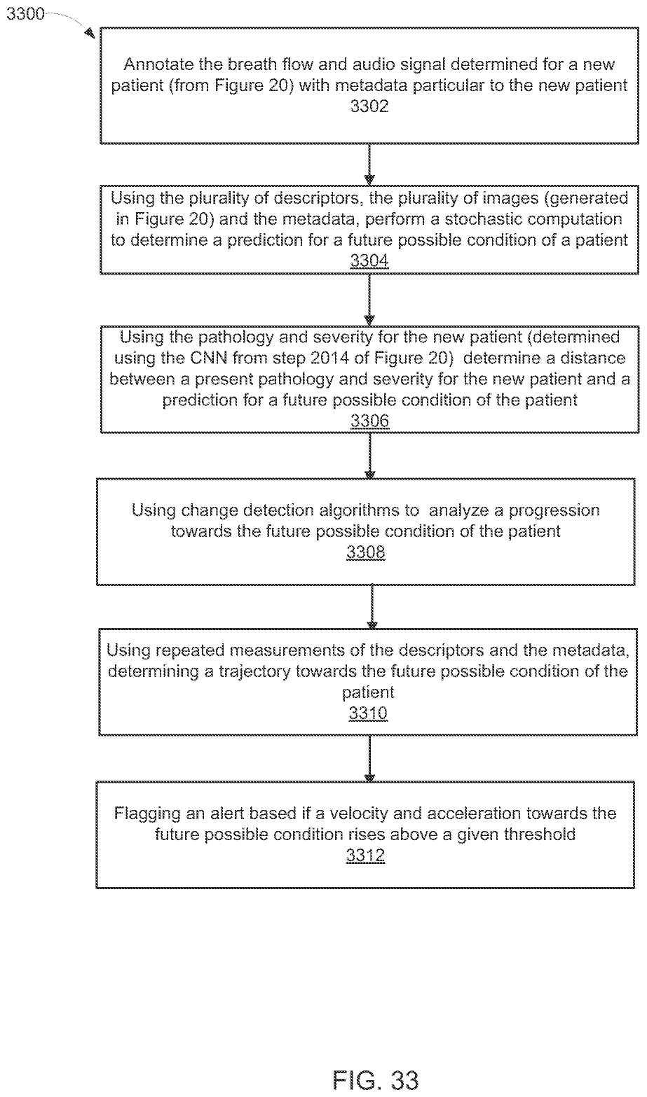

Flow volume loops are graphical representations of a patient's pulmonary function. They are a key component of pulmonary function testing that is ordered for patients who have respiratory conditions (such as asthma or chronic obstructive pulmonary disorder/COPD). FIG. 32 illustrates an exemplary flow volume loop. It should be noted that the flow volume loop is a loop that is cyclic in nature (e.g., things begin at the origin of the graph, travel in the positive X/Y axis directions as the patient is exhaling, then reverse in a route back to the origin to represent inhalation).

The X-axis is measured in the liters of air that is either inspired or expired by the patient. Moving away from the origin on this axis represents exhalation and moving towards the origin on this axis represents inhalation. The Y-axis meanwhile is measured in liters/second, flow is a rate that characterizes how quickly a patient is inspiring/expiring air. Positive values on this axis represent flow out of the lungs (expiration), while negative values on this axis represent flow into the lungs (inspiration).

The greatest utility of a flow-volume loop is that there is a relationship between its shape and the different diseases that affect the lungs. Accordingly, unlike conventional systems, embodiments of the present invention take advantage of both the flow-volume loop computed by the module at block 1803 and the descriptors extracted from the audio recordings to determine lung pathologies and severity. The flow-volume loops and associated flow information (e.g., flow-over-time, volume-over-time) is used in conjunction with the spectrogram and descriptors determined from the time-frequency analysis at block 1825 to train the CNN module 1805.

In one embodiment, a pattern image creation module 1804 is used to create the graphs and images that are used to train the CNN at module 1805. One of the advantages of embodiments of the present invention is that it enables the creation of unique graphs/images (using both flow and sound-based descriptors) that convey information about a patient's pathology and can be used to train the CNN at module 1805. For example, as suggested above, the flow-volume loop determined at block 1803 looks different for patients with different types of pathologies. Accordingly, a flow-volume loop (such as the exemplary one shown in FIG. 32) is one type of image that may be used to train the CNN of module 1805.

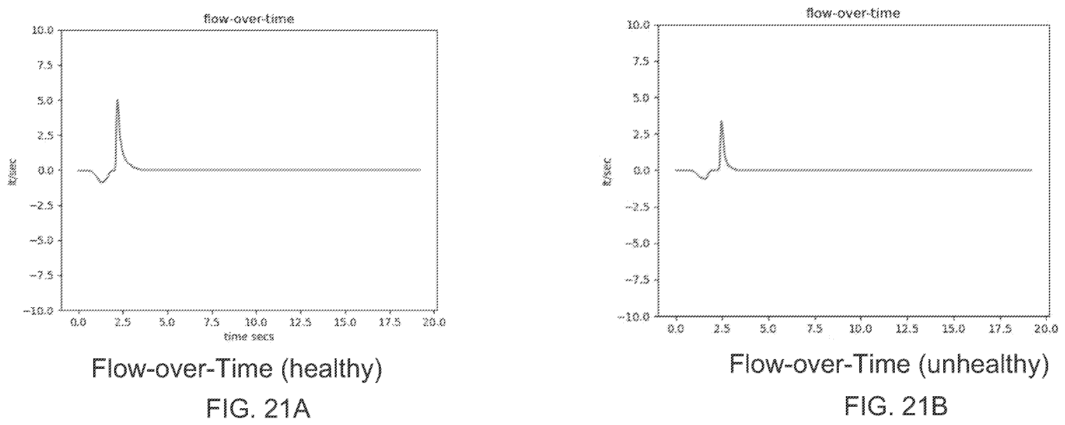

FIGS. 21A, 21B, 22A, 22B, 23A, 23B, 24A, 24B, 25A, 25B, 26A, 26B, 27A, 27B, 28A, 28B, 29A, 29B, 30A, 30B, 31A, 31B (referred to subsequently as the “exemplary graph images”) all provide examples of the types of signal images or graphs that may be used to train the CNN associated with module 1805. Note that the images may be characterized as associated with a “healthy” patient or an “unhealthy” patient based on the annotated metadata or based on the sound-based descriptors computed during time-frequency analysis performed at block 1825. It should be noted that the exemplary graph images may also be characterized as belonging to a “healthy” patient or an “unhealthy” patient by the evaluation module of FIG. 19. In other words, the exemplary graph images may either be annotated graphs that are created by pattern image creation module 1804 and used to train the CNN of module 1805 or they may be images that have been characterized as associated with “healthy” or “unhealthy” patients by the pre-trained CNN module 1904 of FIG. 19 during the evaluation period (as will be discussed further below). The CNN may be trained initially using the exemplary graph images. Subsequent to the training, signal images associated with a new patient may be input to the evaluation framework of FIG. 19 to receive a diagnosis of pathology and severity.

In one embodiment, all the various generated graphs can be synthesized into a pattern image that comprises all of the generated graphs by the pattern image creation module 1804. In other words, all the generated graphs are synthesized into a single image pattern, which is then used to train the CNN.

Referring back to FIG. 18, in one embodiment, the sound-based descriptors extracted from the time-frequency analysis, the flow descriptors (e.g., flow over time, flow volume shape characteristics, etc.) and optionally the metadata (e.g., metadata gathered at blocks 1812, 1814, 1818 and 1824) are used to create graphs/images by module 1804 that together comprise the “training set.” The images used to train the CNN may not only be combinations of flow descriptors and sound-based descriptors, but also unique combinations of sound-based descriptors that are not used by conventional respiratory or flow-based analysis systems.

For example, FIG. 21A is an exemplary illustration of a flow-over-time graph associated with a healthy patient in accordance with an embodiment of the present invention. FIG. 21B is an exemplary illustration of a flow-over-time graph associated with an unhealthy patient in accordance with an embodiment of the present invention. Both figures are associated with purely flow-based descriptors. As can be seen from the figures the peak flow reached by the healthy patient is significantly higher than that reached by an unhealthy patient. In other words, a healthy patient is able to take deeper breaths (with higher commensurate volumes) as compared with unhealthy patients.

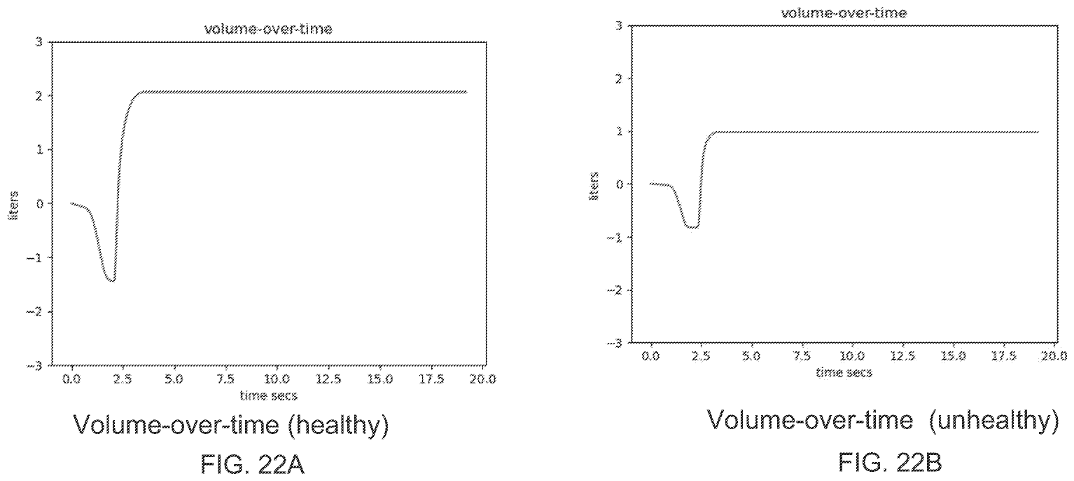

FIG. 22A is an exemplary illustration of a volume-over-time graph associated with a healthy patient in accordance with an embodiment of the present invention. FIG. 22B is an exemplary illustration of a volume-over-time graph associated with an unhealthy patient in accordance with an embodiment of the present invention. The figures are associated with a flow-based descriptor. As can be seen from the figures the peak volume reached by the healthy patient is significantly higher than that reached by an unhealthy patient. In other words, a healthy patient is able to take deeper breaths (with higher commensurate volumes) as compared with unhealthy patients.

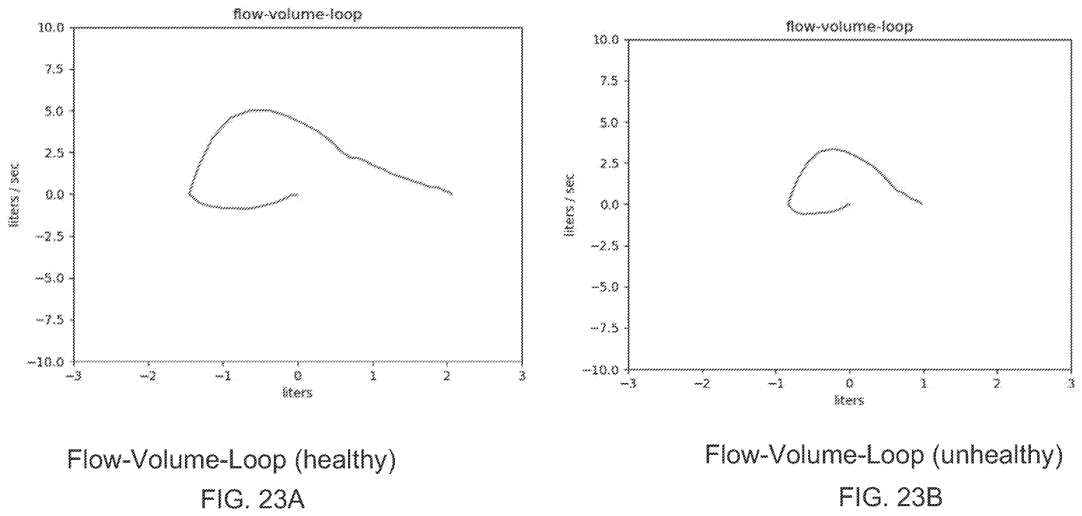

FIG. 23A is an exemplary illustration of a flow-volume loop associated with a healthy patient in accordance with an embodiment of the present invention. FIG. 23B is an exemplary illustration of a flow-volume loop associated with an unhealthy patient in accordance with an embodiment of the present invention. As described above, the shape of a flow-volume loop provides insight into the type of pathology a patient suffers from. In one embodiment, sound-based or other descriptors may be annotated on the flow-volume loop surface to aid an expert in determining a pathology and severity associated with the patient.

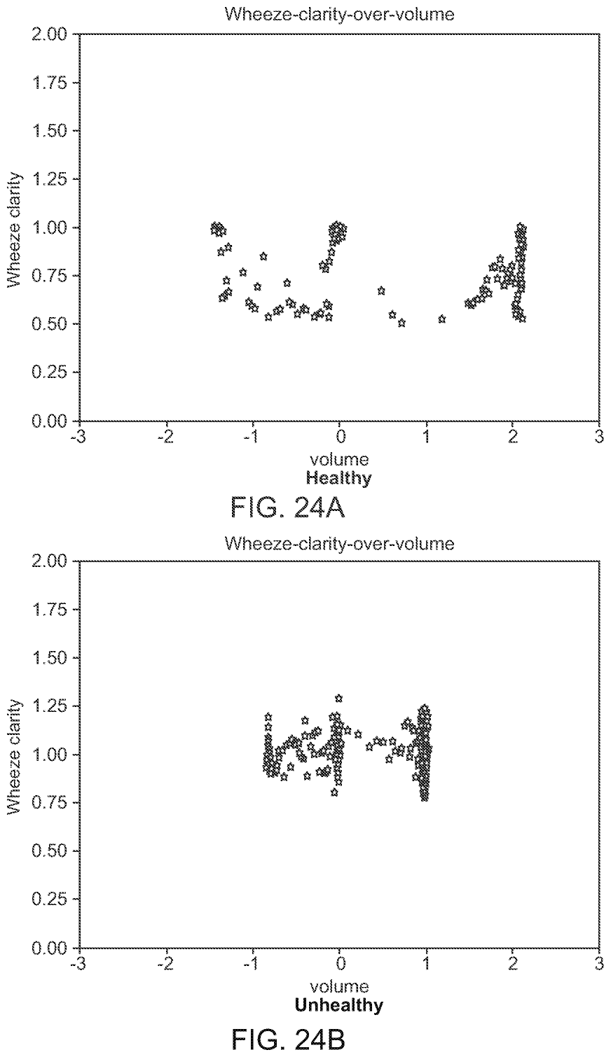

FIG. 24A is an exemplary illustration of a wheeze-clarity—over volume graph associated with a healthy patient in accordance with an embodiment of the present invention. FIG. 24B is an exemplary illustration of a wheeze-clarity over volume graph associated with an unhealthy patient in accordance with an embodiment of the present invention. These graphs are examples of images that are generated by using a combination of sound-based and flow-based descriptors. These images may be used in combination with the other exemplary graph images to train the CNN of FIG. 18. It should be noted that conventional lung pathology diagnosis systems did not use a combination of flow-based and sound-based descriptors to characterize or detect a patient's pathological condition. The pattern creation module 1804 advantageously plots each descriptor in a selected descriptor subset (comprising sound-based and flow-based descriptors) over the rest of the descriptors in the subset. These images are then used to train the CNN associated with module 1805. Creating unique images comprising plots of various descriptors plotted against other related descriptors is a significant improvement over conventional lung pathology analysis systems.

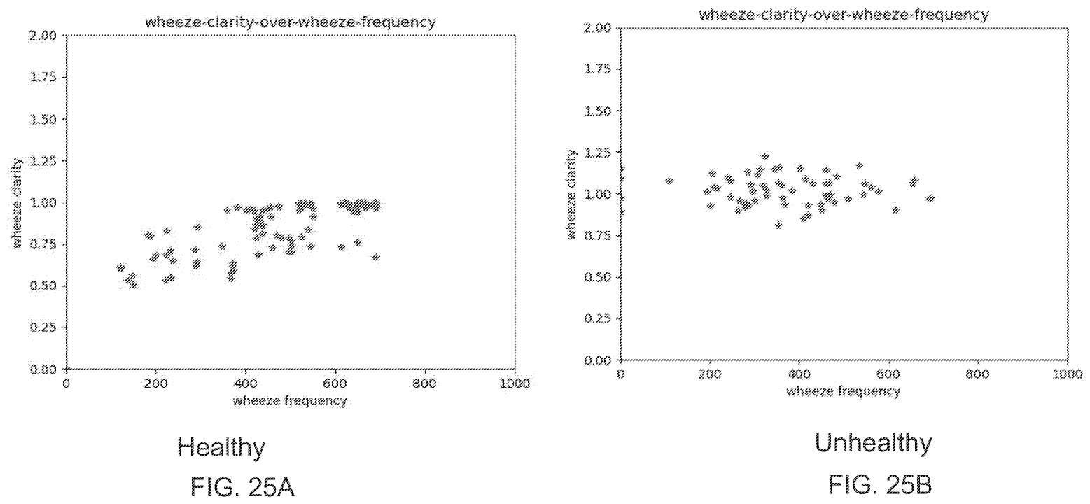

FIG. 25A is an exemplary illustration of a wheeze-clarity over wheeze-frequency graph associated with a healthy patient in accordance with an embodiment of the present invention. FIG. 25B is an exemplary illustration of wheeze-clarity over wheeze-frequency graph associated with an unhealthy patient in accordance with an embodiment of the present invention. These graphs are examples of images that are generated by using a combination of only sound-based descriptors. It should be noted that conventional lung pathology diagnosis systems did not create plots comprising unique combinations of sound-based descriptors to characterize or detect a patient's pathological condition, which were then used to train a CNN. Embodiments of the present invention not only create graphs/images of unique combinations of sound-based descriptors but also use those images to train a CNN to understand differences in the plots between healthy and unhealthy patients. Training the CNN using graphs comprising various plotted combinations of descriptors enables the CNN to learn the visual relationships between the descriptors for healthy and unhealthy patients and, subsequently, use the trained CNN to evaluate a new patient's health condition more efficiently.

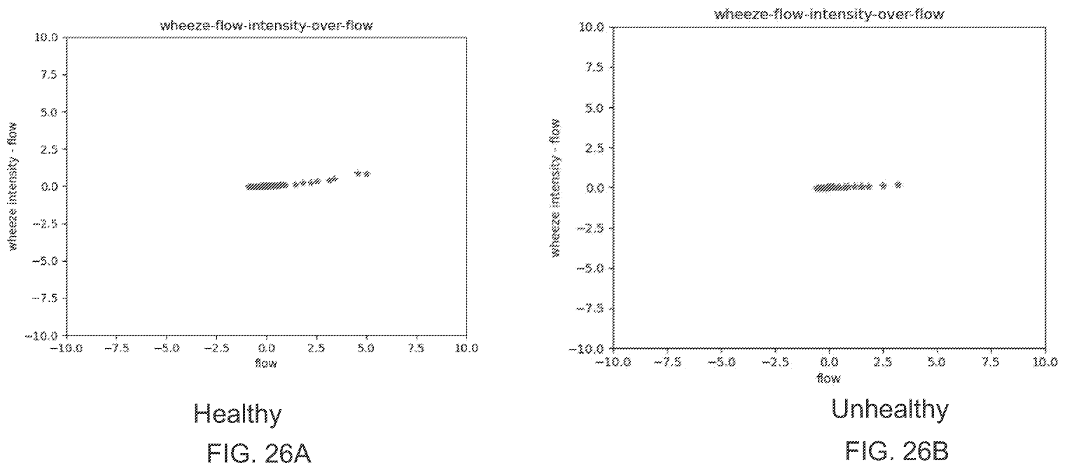

FIG. 26A is an exemplary illustration of a wheeze-flow-intensity over flow graph associated with a healthy patient in accordance with an embodiment of the present invention. FIG. 26B is an exemplary illustration of a wheeze-flow-intensity over flow graph associated with an unhealthy patient in accordance with an embodiment of the present invention. These graphs are examples of images that are generated by using a combination of sound-based and flow-based descriptors. These images may be used in combination with the other exemplary graph images to train the CNN of FIG. 18. It should be noted that conventional lung pathology diagnosis systems did not use a combination of flow-based and sound-based descriptors to characterize or detect a patient's pathological condition. The pattern creation module 1804 advantageously plots each descriptor in a selected descriptor subset (comprising sound-based and flow-based descriptors) over the rest of the descriptors in the subset. These images are then used to train the CNN associated with module 1805. Creating unique images comprising plots of various descriptors plotted against other related descriptors is a significant improvement over conventional lung pathology analysis systems.

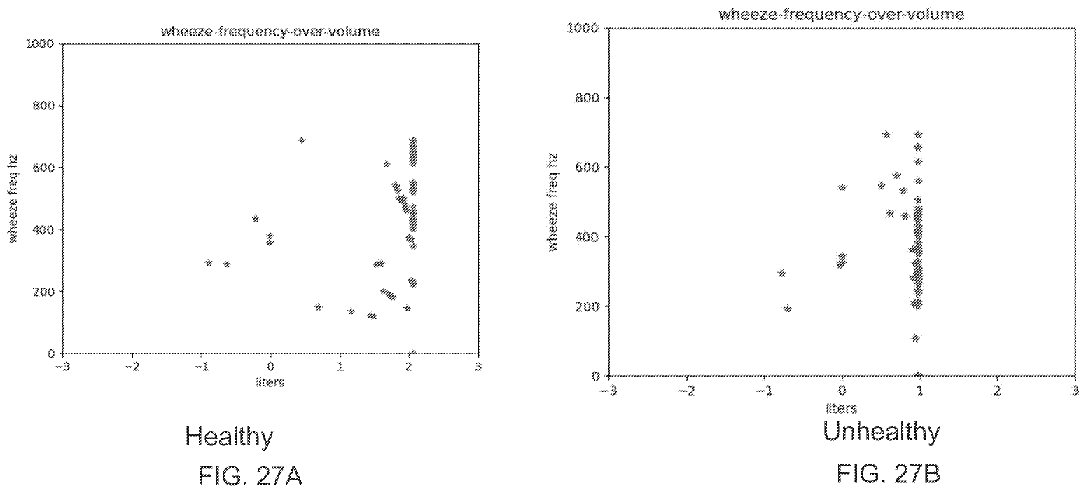

FIG. 27A is an exemplary illustration of a wheeze-frequency over volume associated with a healthy patient in accordance with an embodiment of the present invention. FIG. 27B is an exemplary illustration of a wheeze-frequency over volume graph associated with an unhealthy patient in accordance with an embodiment of the present invention. These graphs are examples of images that are generated by using a combination of sound-based and flow-based descriptors. As mentioned above, a combination of sound-based and flow-based descriptors advantageously allow much richer information to be extracted regarding a patient's pathology and severity than information determined based solely on sound-based descriptors.

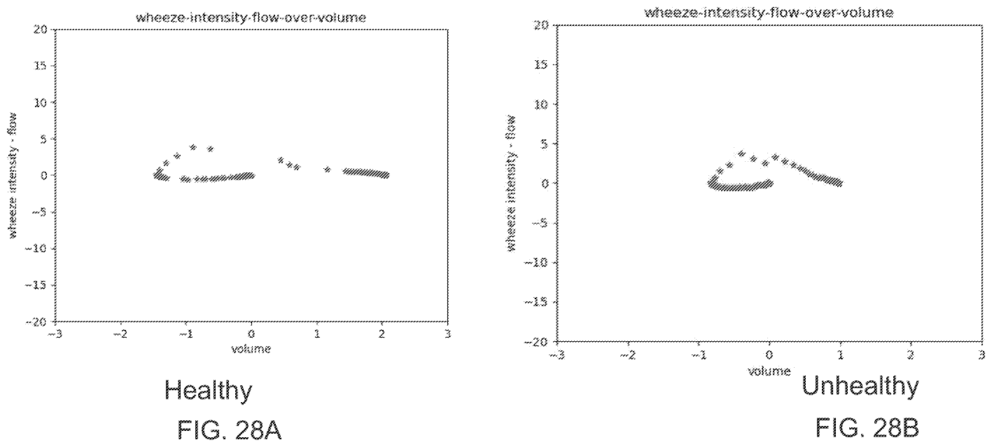

FIG. 28A is an exemplary illustration of a wheeze-intensity-flow over volume associated with a healthy patient in accordance with an embodiment of the present invention. FIG. 28B is an exemplary illustration of a wheeze-intensity-flow over volume graph associated with an unhealthy patient in accordance with an embodiment of the present invention. These graphs are examples of images that are generated by using a combination of sound-based and flow-based descriptors. Wheeze intensity has been discussed as a descriptor. Wheeze intensity-flow is derived from the wheeze intensity descriptor by dividing instantaneous wheeze intensity by instantaneous flow rate at any point in the breath.

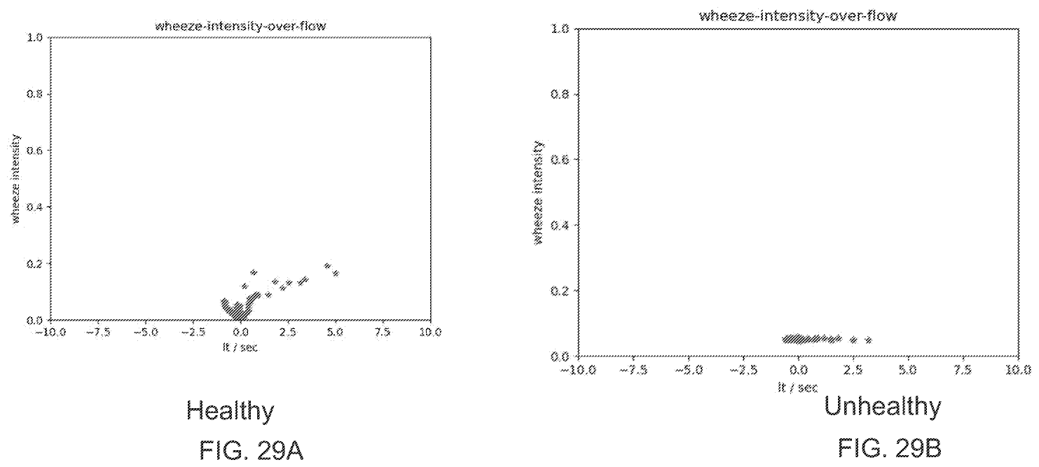

FIG. 29A is an exemplary illustration of a wheeze-intensity over flow associated with a healthy patient in accordance with an embodiment of the present invention. FIG. 29B is an exemplary illustration of a wheeze-intensity over flow graph associated with an unhealthy patient in accordance with an embodiment of the present invention. These graphs are examples of images that are generated by using a combination of sound-based and flow-based descriptors.

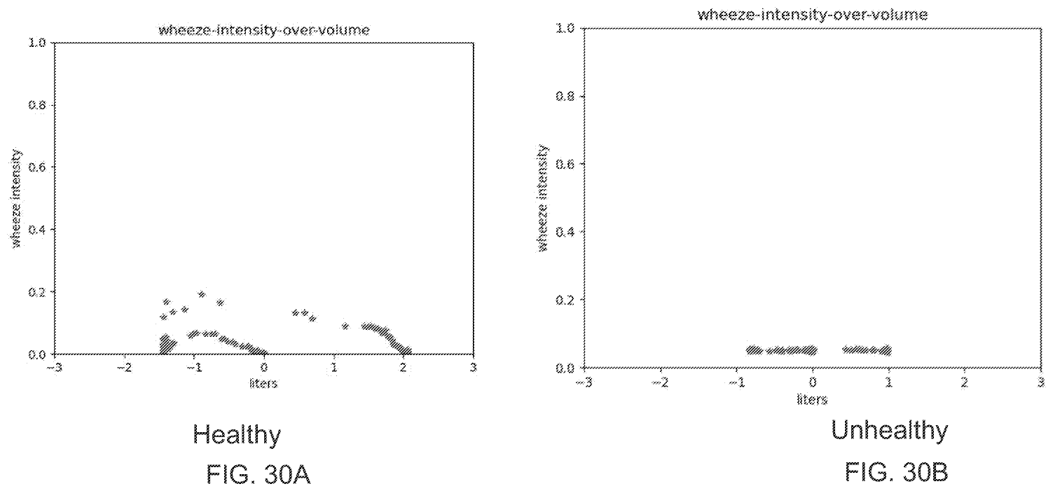

FIG. 30A is an exemplary illustration of a wheeze-intensity over volume associated with a healthy patient in accordance with an embodiment of the present invention. FIG. 30B is an exemplary illustration of a wheeze-intensity over volume graph associated with an unhealthy patient in accordance with an embodiment of the present invention. These graphs are examples of images that are generated by using a combination of sound-based and flow-based descriptors.

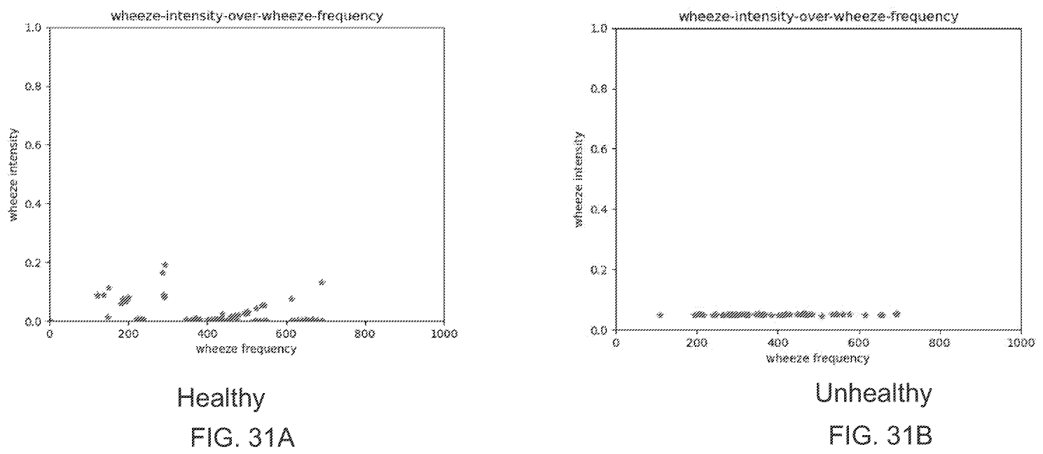

FIG. 31A is an exemplary illustration of a wheeze-intensity over wheeze frequency associated with a healthy patient in accordance with an embodiment of the present invention. FIG. 31B is an exemplary illustration of a wheeze-intensity over wheeze frequency graph associated with an unhealthy patient in accordance with an embodiment of the present invention. These graphs are examples of images that are generated by using a combination of sound-based descriptors. This is another example of images being produced by embodiments of the present invention by plotting descriptors in relation to each other that have not previously been considered by conventional lung analysis systems.

Once the CNN process associated with module 1805 has been fully trained, it may later be used to evaluate new incoming recordings to determine whether they are associated with healthy lungs, and if not, then to determine lung pathology and disease type (e.g., asthma, COPD, etc.) and severity (mild, moderate, severe) as discussed in connection with FIG. 19 below. The classification may include number and letter severity designations per the Global initiative for Obstructive Lung Disease (GOLD) strategy document for the diagnosis management and prevention of COPD. The trained patterns, in one embodiment, along with the metadata (e.g., from blocks 1812, 1818, 1814 and 1824) are stored in a pattern database at block 1806.

In one embodiment, for visual feedback purposes, quantified descriptors or descriptor combinations (either sound-based or flow-based) could be localized over the flow volume loop surface to further aid an expert on the severity/pathology decision for a respiratory session during evaluation. In other words, sound-based or other descriptors may be annotated on the flow-volume loop surface prior to storing in database 1806. The descriptors in combination with the flow-volume curve may assist an expert in diagnosing a patient's condition and severity levels.

In one embodiment, once the CNN has been trained, the next step is to store all the extracted images, spectrograms and descriptors in a database at block 1806. The images/graphs and descriptors may, in one embodiment, be aggregated over pathology and severity to tune the neural network layers and coefficients appropriately. Note also that information associated with any new patient that is evaluated using the evaluation module 1900 of FIG. 19 can also be used to further train or re-tune the CNN.

FIG. 19 illustrates a block diagram providing an overview of the manner in which a convolutional neural network (CNN) can be used to evaluate a respiratory recording associated with a new patient to determine lung pathologies and severity in accordance with an embodiment of the present invention.

The evaluation or decision-making module 1900 shown in FIG. 19 receives as an input a recording from a new patient at block 1919. The evaluation module then applies time-frequency analysis and extracts a spectrogram at block 1402. This is substantially similar to the manner in which a spectrogram is extracted at block 1402 in FIG. 14 and to the manner in which the spectrogram is extracted at block 1809 in FIG. 18.

In one embodiment, one or more Probably Density Functions (PDFs) may also be extracted at block 1951 and binary hypothesis testing may be conducted similar to the manner discussed in connection with FIG. 14. As noted above, the PDFs can be used in the evaluation module (discussed in connection with FIG. 19) to decide if a new respiratory recording that is being evaluated by the AI module belongs to a healthy category or to a category indicating disease by employing a Binary Hypothesis Likelihood Ratio Test (discussed in connection with FIG. 16) (BHLRT). As noted previously, the BHLRT may be used to characterize a patient's condition as “healthy” or “unhealthy.”

Similar to FIG. 14, the evaluation module of FIG. 19 also comprises a module 1970 for sound-based wheeze description extraction, a module 1907 for sound-based airflow analogous descriptor extraction, a module 1917 for determining wheeze sources and a module 1988 for determining crackling descriptors. These modules perform substantially the same function as their corresponding counterparts in FIG. 14.

Meanwhile, simultaneous with the audio input recording at block 1919, a spirometer (or other device or devices that can capture audio and flow information together or separately) captures breath flow data at block 1901 that is synced to the input respiratory audio recording at block 1902. Both the audio input recording and the breath flow data may be received in the form of data files comprising the audio and flow-based signals. From the flow data, volume over time, flow over time, and flow-volume loop information is determined by module 1903. The graphs associated with volume over time, flow over time and flow-volume loop were discussed in connection with FIGS. 21A, 21B, 22A, 22B, 23A and 23B.

The time-frequency data (and descriptors and spectrograms associated therewith) from module 1925 and the flow data (from module 1903) are inputted to a pattern creation module 1904. Similar to the pattern creation module 1804, the flow and sound-based descriptors are used by pattern image creation module 1904 to generate one or more graphs or images that can be transmitted to the CNN pattern image deep learning and classification module 1905 for evaluation. In one embodiment, all the various generated graphs can be synthesized into a pattern image that comprises all of the generated graphs.

The evaluation system then determines the pathology, disease and severity at module 1906 using the information learned from the processing of the training sets. Note that the metadata from blocks 1984, 1982, 1983 and 1992 (which is substantially similar to the metadata from blocks 1812, 1818, 1814 and 1824 in FIG. 18) can also be used at module 1906 to aid in the diagnosis of patients and the severity of their condition. Further note that in one embodiment, module 1906 may be a rule-based/fuzzy logic module that combines the output from the CNN with other patient data and metadata to provide a final result into pathology and severity.

As mentioned above, the metadata may include other physiological measurements and diagnostics, including pulmonary function testing (spirometry), blood oxygen levels (pulse oximetry), respiratory gas analysis (O2, CO2, VOCs, FeNO), body temperature, plethsmography, CT scans, and blood and sputum inflammatory. genetic markers can also be fed into the CNN processes or used in conjunction with the CNN output to determine patient pathology and severity. There are 2 distinct types of patient data. Data related to birth date, height, weight and gender and data related to patient health and care events such as exacerbation events and hospitalizations. Patient data may be pulled form electronic health records automatically without human intervention. Medication usage and tracking, a users' symptoms, exercise and diet, and a doctor's diagnosis, can also be fed into the CNN process or used in conjunction with the CNN output to determine patient pathology and severity.

In one embodiment, for visual feedback purposes, quantified descriptors or descriptor combinations can also be localized over or used to annotate the flow-volume loop surface to further aid an expert on the severity and pathology determination for a respiratory session. The descriptors and metrics can be localized on the flow-volume plane and help determined both the kind of pathology and its severity, as well as provide meaningful metadata for tracking purposes and visual assistive feedback. This information can then be inputted back, using a feedback loop, into the training module of FIG. 18. The training module can then make determinations on the severity of subsequent sessions. In one embodiment, multiple annotated sessions are used to train the CNN, which after training is over is ready to evaluate images/graphs associated with new patients.

After the new patient session is classified at module 1906, it is subsequently stored to the training database at module 1907 in order to augment the training set. Subsequently, the process re-runs the training to update its state at block 1999. The extracted features may also be stored to the user profile database in order to compare the new user data to the previous user data for tracking purposes. If a new recording shows characteristic of pathology or disease progression, its characteristics can be compared to the data that has been extracted from older recordings in order to estimate the rate of pathology or disease progression.

In one embodiment, the CNN can also be trained periodically with new sessions that an expert (e.g., doctor) may annotate to keep it updated and allow it to become more personalized, e.g., in circumstances where only a single patient's data is used. The CNN may also keep improving in accuracy and robustness since it will be periodically retrained with new data that an expert will typically have annotated correctly (following a semi supervised scheme). Further, the evaluation module of FIG. 19 may have the potential and the flexibility to be personalized if this is desirable.

In this way, embodiments of the present invention provide a framework and processes for determining pathologies that are optimized for determining the manner in which a condition or a pathology is trending over time. Accordingly, a deeper understanding of how a pathology is responding to treatments or changing over time is made available.

In one embodiment, the sound-based and flow-based descriptors and/or graphs/images computed may be used to further compute stochastic distributions that correspond to predictions regarding a patient's future state (e.g., a possible future decline in a patient's healthy or an increase in the severity of their condition). For example, a stochastic computation may show that the flow-volume loop exhalation curve is shaped towards a faster, steeper and more exponential decay that would correspond to a worse state of health than the current one. In one embodiment, the stochastic computation may be semi-personalized, e.g., the CNN data may be used to create possible “future” images or graphs corresponding to a patient based on information related to prior patients that was used to train the CNN. For example, prior patients' age, race, gender, medical history, etc. may be used to create future projections about a new patient.

In one embodiment, the fuzzy logic/rule-based module 1906 of FIG. 19 also performs a quantification of a patient's health status based on selected descriptors (e.g., calculation of descriptor-based scores) and calculation of the distance that a particular combination of descriptors has from the pre-calculated future patient image computed as part of the stochastic prediction discussed above. Repeated measurements would recalculate these scores and distances from the simulated future image, and would create a trajectory towards the future image, e.g., with a velocity and an acceleration that would be statistically analyzed by change detection processes. Furthermore, module 1906 may also be programmed to raise an alert if necessary, warning the patient, the caregiver and the doctor about a possible upcoming exacerbation.

FIG. 20 depicts a flowchart 2000 illustrating an exemplary computer-implemented process for determining lung pathologies and severity from a respiratory recording and breath flow analysis using a convolutional neural network in accordance with one embodiment of the present invention. While the various steps in this flowchart are presented and described sequentially, one of ordinary skill will appreciate that some or all of the steps can be executed in different orders and some or all of the steps can be executed in parallel. Further, in one or more embodiments of the invention, one or more of the steps described below can be omitted, repeated, and/or performed in a different order. Accordingly, the specific arrangement of steps shown in FIG. 20 should not be construed as limiting the scope of the invention. Rather, it will be apparent to persons skilled in the relevant art(s) from the teachings provided herein that other functional flows are within the scope and spirit of the present invention. Flowchart 2000 may be described with continued reference to exemplary embodiments described above, though the method is not limited to those embodiments.

At step 2002, a plurality of audio respiratory and breath flow signals are received, wherein the signals comprise a training set to be processed and inputted into a convolutional neural network (CNN). The plurality of audio and breath flow signals comprise sessions with patients with known pathologies of varying degrees of severity.

At step 2004, the plurality of audio signals are synchronized with the plurality of breath flow signals.

At step 2006, the plurality of audio and breath flow signals are analyzed to extract a plurality of descriptors associated with both the audio and the breath flow.

At step 2008, a plurality of images (or graphs) are created and stored in computer memory using information from the descriptors associated with the respiratory audio and breath flow signals, wherein at least one of the images comprises a plot that is a combination of descriptors from both the respiratory audio and flow signals. Note that the other images may be a plot of the breath flow signals over time or a plot of a unique combination of other sound-based descriptors.

At step 2010, training for the CNN is performed using the plurality of images. In one embodiment, the images may be annotated with metadata relevant to the patients and the known pathologies. For example, the metadata used to annotate the respiratory recordings may comprise respiratory measurements and diagnostics 1812 (spirometry, plethysmography, inflammatory markers, ventilation, CT scans, auscultation, etc.), medication 1818, patient symptoms 1814, and doctor's diagnoses 1824. Other physiological measurements and diagnostics, including pulmonary function testing (spirometry), blood oxygen levels (pulse oximetry), respiratory gas analysis (O2, CO2, VOCs, FeNO), body temperature, and blood and sputum inflammatory and genetic markers can also be annotated on the images and fed to the CNN. In addition, medication usage and tracking, users' symptoms, exercise and diet habits, air quality, and a doctor's diagnosis, can also be fed into the CNN process.

At step 2012, at least one image is created for a new patient using a breath flow signal and an audio respiratory signal associated with the new patient and inputted into the CNN

At step 2014, the CNN is used to determine a pathology and associated severity for the new patient. In one embodiment, the metadata discussed above may be used in conjunction with the result from the CNN to aid in the determination of the pathology and severity.

At step 2016, the CNN is updated with at least one image and associated metadata for the new patient and re-tuned using the new patient data.

FIG. 33 depicts a flowchart 3300 illustrating an exemplary computer-implemented process for determining lung pathologies and severity from a respiratory recording and breath flow analysis using a convolutional neural network in accordance with one embodiment of the present invention. While the various steps in this flowchart are presented and described sequentially, one of ordinary skill will appreciate that some or all of the steps can be executed in different orders and some or all of the steps can be executed in parallel. Further, in one or more embodiments of the invention, one or more of the steps described below can be omitted, repeated, and/or performed in a different order. Accordingly, the specific arrangement of steps shown in FIG. 33 should not be construed as limiting the scope of the invention. Rather, it will be apparent to persons skilled in the relevant art(s) from the teachings provided herein that other functional flows are within the scope and spirit of the present invention. Flowchart 3300 may be described with continued reference to exemplary embodiments described above, though the method is not limited to those embodiments.

At block 3302, the audio and breath flow signals determined for a new patient at block 2012 in FIG. 20 are annotated with metadata particular to the new patient.

At block 3304, using the plurality of descriptors and the plurality of images determined (e.g., at blocks 2006, 2008 and 2012 of FIG. 20) along with the metadata at block 3302 and potentially previous results from individuals or populations with similar demographics or the like, a stochastic computation is performed to determine a prediction for a future possible condition of a patient.

At block 3306, using the pathology and severity for the new patient (determined, for example, using the CNN at block 2014 of FIG. 20) a distance is determined between the future possible condition of the patient and the existing pathology and severity of the new patient. As mentioned above, in one embodiment, the metadata, the sound-based and flow-based descriptors and/or graphs/images computed may be used to further compute stochastic distributions that correspond to predictions regarding a patient's future state (e.g., a possible future decline in a patient's healthy or an increase in the severity of their condition). For example, a stochastic computation may show that the flow-volume loop exhalation curve is shaped towards a faster, steeper and more exponential decay that would correspond to a worse state of health than the current one. In one embodiment, the stochastic computation may be semi-personalized, e.g., the CNN data may be used to create possible “future” images or graphs corresponding to a patient based on information related to prior patients that was used to train the CNN. For example, prior patients' age, race, gender, medical history, etc. may be used to create future projections about a new patient. In another embodiment the trend may be that of a population or a social group. The trends of different social groups may be compared.

At block 3308, change detection algorithms may be used to analyze the progression of the pathology associated with the new patient towards the predicted future possible condition of the patient or group.

At block 3310, in one embodiment, repeated measurements for the patient would recalculate these scores and distances from the simulated future image. Further repeated measurements can be used to create a trajectory towards the simulated future image with a velocity and an acceleration that would be statistically analyzed by change detection algorithms. Given sufficient data future paths might be simulated under different care conditions.

At block 3312, the change detection algorithms would raise an alert if necessary, warning the patient, the caregiver and the doctor about a possible upcoming exacerbation if the velocity and the acceleration rise above a predetermined threshold.

While the foregoing disclosure sets forth various embodiments using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered as examples because many other architectures can be implemented to achieve the same functionality.

The process parameters and sequence of steps described and/or illustrated herein are given by way of example only. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. These software modules may configure a computing system to perform one or more of the example embodiments disclosed herein. One or more of the software modules disclosed herein may be implemented in a cloud computing environment. Cloud computing environments may provide various services and applications via the Internet. These cloud-based services (e.g., software as a service, platform as a service, infrastructure as a service, etc.) may be accessible through a Web browser or other remote interface. Various functions described herein may be provided through a remote desktop environment or any other cloud-based computing environment.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as may be suited to the particular use contemplated.

Embodiments according to the invention are thus described. While the present disclosure has been described in particular embodiments, it should be appreciated that the invention should not be construed as limited by such embodiments, but rather construed according to the below claims.

What is claimed is:

1. A computer-implemented method for determining lung pathology from a subject under test, the method comprising:
receiving a training set comprising a plurality of breath flow signals and a plurality of audio signals for a convolutional neural network, wherein the training set is extracted from sessions with subjects with known pathologies of known degrees of severity;
synchronizing each of the plurality of breath flow signals with a corresponding one of the plurality of audio signals;
analyzing the plurality of audio signals and the plurality of breath flow signals to extract a plurality of descriptors associated with the plurality of audio signals and the plurality of breath flow signals;
creating a plurality of graphs in computer readable memory using information from the plurality of descriptors, wherein at least one of the graphs comprises a plot combining descriptors from both the plurality of audio signals and the plurality of breath flow signals;
training the convolutional neural network using the plurality of graphs;
creating at least one test graph using a breath flow signal and an audio signal from the subject under test;
inputting the at least one test graph associated with the subject under test into the convolutional neural network; and
determining a pathology and associated severity for the subject under test using the convolutional neural network.

2. The method of claim 1, further comprising:
updating the training set with the at least one test graph associated with the subject under test; and
repeating the training of the convolutional neural network with the training set as updated by the updating.

3. The method of claim 1, wherein a subset of the plurality of descriptors is associated with the plurality of breath flow signals and is selected from a group consisting of: flow over time descriptors, flow over volume descriptors and flow volume loop descriptors.

4. The method of claim 1, wherein the creating the plurality of graphs further comprises annotating the plurality of graphs with metadata, wherein the metadata is selected from a group consisting of: health status; pathology; results from diagnostic tests; severity of pathology; respiratory measurements and diagnostics; inflammatory markers; CT scans; auscultation; pulmonary function testing; blood oxygen levels; respiratory gas analysis; body temperature; blood and sputum inflammatory and genetic markers; medication usage; patient's symptoms; air quality; and exercise and diet habits.

5. The method of claim 1, wherein a subset of the plurality of descriptors is associated with the plurality of audio signals and is selected from a group consisting of: wheeze-based descriptors, crackling-based descriptors and sound-based airflow analogous descriptors.

6. The method of claim 1, wherein the training set is captured by a spirometer comprising a flow sensor and a microphone.

7. The method of claim 1, further comprising:
determining a prediction for a future condition of the subject under test by using a stochastic process to evaluate the plurality of descriptors.

8. The method of claim 1, wherein the creating the plurality of graphs comprises synthesizing the plurality of graphs into a consolidated pattern image.

9. A non-transitory computer-readable storage medium having stored thereon, computer executable instructions that, if executed by a computer system cause the computer system to perform a method determining lung pathology for a subject under test the method comprising:
receiving a training set comprising a plurality of breath flow signals and a plurality of audio signals for a convolutional neural network, wherein the training set is extracted from sessions with subjects with known pathologies of known degrees of severity;
consolidating each of the plurality of breath flow signals with a corresponding one of the plurality of audio signals to produce a synchronized training set;
analyzing the synchronized training set by extracting a plurality of descriptors therefrom;
creating a plurality of graphs resident in computer memory using information from the plurality of descriptors, wherein at least one of the graphs comprises a plot combining descriptors from both the plurality of audio signals and the plurality of breath flow signals;
training the convolutional neural network using the plurality of graphs;
creating at least one test graph using a breath flow signal and an audio signal from a subject under test;
inputting the at least one test graph associated with the subject under test into the convolutional neural network; and
determining a pathology and associated severity for the subject under test using the convolutional neural network.

10. The non-transitory computer-readable storage medium of claim 9, wherein the method further comprises:
updating the training set with the at least one test graph associated with the subject under test; and
repeating the training of the convolutional neural network with the training set as updated by the updating.

11. The non-transitory computer-readable storage medium of claim 9, wherein a subset of the plurality of descriptors is associated with the plurality of breath flow signals and is selected from a group consisting of: flow over time descriptors, flow over volume descriptors and flow volume loop descriptors.

12. The non-transitory computer-readable storage medium of claim 9, wherein the creating the plurality of graphs further comprises annotating the plurality of graphs with metadata, wherein the metadata is selected from a group consisting of: health status; pathology; results from diagnostic tests; severity of pathology; respiratory measurements and diagnostics; inflammatory markers; CT scans; auscultation; pulmonary function testing; blood oxygen levels; respiratory gas analysis; body temperature; blood and sputum inflammatory and genetic markers; medication usage; patient's symptoms; air quality; and exercise and diet habits.

13. The non-transitory computer-readable storage medium of claim 9, wherein a subset of the plurality of descriptors is associated with the plurality of audio signals and is selected from a group consisting of: wheeze-based descriptors, crackling-based descriptors and sound-based airflow analogous descriptors.

14. The non-transitory computer-readable storage medium of claim 9, further comprising capturing the training set using a spirometer comprising a flow sensor and a microphone.

15. The non-transitory computer-readable storage medium of claim 9, wherein the creating the plurality of graphs comprises synthesizing the plurality of graphs into a consolidated pattern image.

16. A spirometer system for determining lung pathology from breath flow and audio respiratory signals, the spirometer system comprising:
a memory for storing a plurality of audio signals and a plurality of breath flow signals, instructions associated with a convolutional neural network and a process for determining lung pathology from the plurality of audio signals and the plurality of breath flow signals;
a processor coupled to the memory, the processor being configured to operate in accordance with the instructions to:
receive a plurality of breath flow signals and a plurality of audio signals comprising a training set for a convolutional neural network, wherein the plurality of breath flow signals and the plurality of audio signals are extracted from sessions with persons with known pathologies of known degrees of severity;
synchronize each of the plurality of breath flow signals with a corresponding one of the plurality of audio signals;
analyze the plurality of audio signals and the plurality of breath flow signals to extract a plurality of descriptors associated with the plurality of audio signals and the plurality of breath flow signals;
create a plurality of graphs using information from the plurality of descriptors, wherein at least one of the graphs comprises a plot combining descriptors from both the plurality of audio signals and the plurality of breath flow signals;
train the convolutional neural network using the plurality of graphs;
create at least one test graph using a breath flow signal and an audio signal from a patient;
input the at least one test graph associated with the patient into the convolutional neural network; and
determine a pathology and associated severity for the patient using the convolutional neural network.

17. The spirometer system of claim 16, wherein the processor is further configured to operate in accordance with the instructions to:
update the training set with the at least one test graph associated with the new patient; and
repeat the training of the convolutional neural network with the updated training set updated by the updating.

18. The spirometer system of claim 16, wherein a subset of the plurality of descriptors is associated with the plurality of breath flow signals and is selected from a group consisting of: flow over time descriptors, flow over volume descriptors and flow volume loop descriptors.

19. The spirometer system of claim 16, wherein a subset of the plurality of descriptors is associated with the plurality of audio signals and is selected from a group consisting of: wheeze-based descriptors, crackling-based descriptors and sound-based airflow analogous descriptors.

20. The system of claim 16, wherein the spirometer system comprises a dual-sense spirometer comprising a flow sensor to capture the plurality of breath flow signals and a microphone to capture the plurality of audio signals.
